SEQUENCE LISTING (66 sequences)

SEQ ID NO: 1 - DNA, 21 bp, Artificial Sequence
  Cdh1 FW primer
cagttccgaggtctacacctt

SEQ ID NO: 2 - DNA, 22 bp, Artificial Sequence
  Cdh1 REV primer
tgaatcgggagtcttccgaaaa

SEQ ID NO: 3 - DNA, 21 bp, Artificial Sequence
  Cldn1 FW primer
tgccccagtggaagatttact

SEQ ID NO: 4 - DNA, 20 bp, Artificial Sequence
  Cldn1 REV primer
ctttgcgaaacgcaggacat

SEQ ID NO: 5 - DNA, 21 bp, Artificial Sequence
  Cldn2 FW primer
caactggtgggctacatccta

SEQ ID NO: 6 - DNA, 19 bp, Artificial Sequence
  Cldn2 REV primer
cccttggaaaagccaaccc

SEQ ID NO: 7 - DNA, 21 bp, Artificial Sequence
  Cldn3 FW primer
accaactgcgtacaagacgag

SEQ ID NO: 8 - DNA, 20 bp, Artificial Sequence
  Cldn3 REV primer
cgggcaccaacgggttatag

SEQ ID NO: 9 - DNA, 19 bp, Artificial Sequence
  Cldn5 FW primer
gcaaggtgtatgaatctgt

SEQ ID NO: 10 - DNA, 22 bp, Artificial Sequence
  Cldn5 REV primer
gtcaaggtaacaaagagtgcca

SEQ ID NO: 11 - DNA, 19 bp, Artificial Sequence
  Cldn7 FW primer
ggcctgatagcgagcactg

SEQ ID NO: 12 - DNA, 21 bp, Artificial Sequence
  Cldn7 REV primer
tggcgacaaacatggctaaga

SEQ ID NO: 13 - DNA, 21 bp, Artificial Sequence
  Cldn15 FW primer
attgcagggaccctccacata

SEQ ID NO: 14 - DNA, 21 bp, Artificial Sequence
  Cldn15 REV primer
gcccagttcatacttggttcc

SEQ ID NO: 15 - DNA, 21 bp, Artificial Sequence
  Crb3 FW primer
caccggaccctttcacaaata

SEQ ID NO: 16 - DNA, 21 bp, Artificial Sequence
  Crb3 REV primer
cccactgctataaggaggact

SEQ ID NO: 17 - DNA, 21 bp, Artificial Sequence
  Dlg1 FW primer
agtgacgaagtcggagtgatt

SEQ ID NO: 18 - DNA, 22 bp, Artificial Sequence
  Dlg1 REV primer
gtcagggatctcccctttatct

SEQ ID NO: 19 - DNA, 23 bp, Artificial Sequence
  Jam1 FW primer
tctcttcacgtctatgatcctgg

SEQ ID NO: 20 - DNA, 21 bp, Artificial Sequence
  Jam1 REV primer
tttgatggactcgttctgggg

SEQ ID NO: 21 - DNA, 22 bp, Artificial Sequence
  Jam2 FW primer
gtgcccacttctgttatgactg

SEQ ID NO: 22 - DNA, 21 bp, Artificial Sequence
  Jam2 REV primer
ttccctagcaaacttgtgcca

SEQ ID NO: 23 - DNA, 19 bp, Artificial Sequence
  Jam3 FW primer
ctgcgacttcgactgtacg

SEQ ID NO: 24 - DNA, 22 bp, Artificial Sequence
  Jam3 REV primer
ttcggttgctggatttgagatt

SEQ ID NO: 25 - DNA, 19 bp, Artificial Sequence
  Llgl1 FW primer
gcttccccaatcagcccag

SEQ ID NO: 26 - DNA, 21 bp, Artificial Sequence
  Llgl1 REV primer
gcgcagccattatgatggatg

SEQ ID NO: 27 - DNA, 24 bp, Artificial Sequence
  Muc1 FW primer
ggttgctttggctatcgtctattt

SEQ ID NO: 28 - DNA, 21 bp, Artificial Sequence
  Muc1 REV primer
aaagatgtccagctgcccata

SEQ ID NO: 29 - DNA, 20 bp, Artificial Sequence
  Muc2 FW primer
atgcccacctcctcaaagac

SEQ ID NO: 30 - DNA, 23 bp, Artificial Sequence
  Muc2 REV primer
gtagtttccgttggaacagtgaa

SEQ ID NO: 31 - DNA, 22 bp, Artificial Sequence
  Muc4 FW primer
acaggtgtaactagaagcctcg

SEQ ID NO: 32 - DNA, 21 bp, Artificial Sequence
  Muc4 REV primer
caggggtgctatgcactactg

SEQ ID NO: 33 - DNA, 21 bp, Artificial Sequence
  Muc13 FW primer
gccagtcctcccaccacggta

SEQ ID NO: 34 - DNA, 21 bp, Artificial Sequence
  Muc13 REV primer
ctgggacctgtgcttccaccg

SEQ ID NO: 35 - DNA, 21 bp, Artificial Sequence
  Mylk FW primer
tgggggacgtgaaactgtttg

SEQ ID NO: 36 - DNA, 19 bp, Artificial Sequence
  Mylk REV primer
ggggcagaatgaaagctgg

SEQ ID NO: 37 - DNA, 22 bp, Artificial Sequence
  Ocln FW primer
ggcggatatacagacccaagag

SEQ ID NO: 38 - DNA, 24 bp, Artificial Sequence
  Ocln REV primer
gataatcatgaaccccaggacaat

SEQ ID NO: 39 - DNA, 20 bp, Artificial Sequence
  Pals1 FW primer
tttgggcaccagaatgatgc

SEQ ID NO: 40 - DNA, 23 bp, Artificial Sequence
  Pals1 REV primer
aacaattccttcttccgtgtcaa

SEQ ID NO: 41 - DNA, 21 bp, Artificial Sequence
  Par3 FW primer
ggagatggccgcatgaaagtt

SEQ ID NO: 42 - DNA, 21 bp, Artificial Sequence
  Par3 REV primer
ctccaagcgatgcacctgtat

SEQ ID NO: 43 - DNA, 20 bp, Artificial Sequence
  Par6 FW primer
tcagaaacgggcagaaggtg

SEQ ID NO: 44 - DNA, 20 bp, Artificial Sequence
  Par6 REV primer
ccaggcgggagatgaagata

SEQ ID NO: 45 - DNA, 21 bp, Artificial Sequence
  Patj FW primer
ttcgatgggcaccactatatc

SEQ ID NO: 46 - DNA, 21 bp, Artificial Sequence
  Patj REV primer
ggtgggggcacttctttaagg

SEQ ID NO: 47 - DNA, 21 bp, Artificial Sequence
  aPkc lambda FW primer
cactttgagccttccatctcc

SEQ ID NO: 48 - DNA, 19 bp, Artificial Sequence
  aPkc lambda REV primer
gtgaccagcttgtggcact

SEQ ID NO: 49 - DNA, 21 bp, Artificial Sequence
  aPkc sigma FW primer
gcgtggatgccatgacaacat

SEQ ID NO: 50 - DNA, 21 bp, Artificial Sequence
  aPkc sigma REV primer
ggctcttgggaaggcatgaca

SEQ ID NO: 51 - DNA, 21 bp, Artificial Sequence
  Rpl4 FW primer
ccgtcccctcatatcggtgta

SEQ ID NO: 52 - DNA, 23 bp, Artificial Sequence
  Rpl4 REV primer
gcatagggctgtctgttgttttt

SEQ ID NO: 53 - DNA, 20 bp, Artificial Sequence
  Scrib FW primer
cctgggcatcagtatcgcag

SEQ ID NO: 54 - DNA, 20 bp, Artificial Sequence
  Scrib REV primer
gccctcgtcatctcctttgt

SEQ ID NO: 55 - DNA, 21 bp, Artificial Sequence
  Tjp1 FW primer
gagcgggctaccttactgaac

SEQ ID NO: 56 - DNA, 23 bp, Artificial Sequence
  Tjp1 REV primer
gtcatctctttccgaggcattag

SEQ ID NO: 57 - DNA, 20 bp, Artificial Sequence
  Tjp2 FW primer
atgggagcagtacaccgtga

SEQ ID NO: 58 - DNA, 22 bp, Artificial Sequence
  Tjp2 REV primer
tgaccaccctgtcattttcttg

SEQ ID NO: 59 - DNA, 21 bp, Artificial Sequence
  Tjp3 FW primer
ctgtggagaacgtcacatctg

SEQ ID NO: 60 - DNA, 20 bp, Artificial Sequence
  Tjp3 REV primer
cggggacgcttcactgtaac

SEQ ID NO: 61 - DNA, 73 bp, Artificial Sequence
  dt_BC1001_PB
  unsure (73)..(73): /note=“v is A or C or G” /note=“n is any nucleotide”
  misc_feature (73)..(73): n is a, c, g, or t
aagcagtggtatcaacgcagagtaccacatatcagagtgcgttttttttttttttttttttttttttttttvn

SEQ ID NO: 62 - DNA, 73 bp, Artificial Sequence
  dt_BC1002_PB
  unsure (73)..(73): /note=“v is A or C or G” /note=“n is any nucleotide”
  misc_feature (73)..(73): n is a, c, g, or t
aagcagtggtatcaacgcagagtacacacacagactgtgagttttttttttttttttttttttttttttttvn

SEQ ID NO: 63 - DNA, 73 bp, Artificial Sequence
  dt_BC1003_PB
  unsure (73)..(73): /note=“v is A or C or G” /note=“n is any nucleotide”
  misc_feature (73)..(73): n is a, c, g, or t
aagcagtggtatcaacgcagagtacacacatctcgtgagagttttttttttttttttttttttttttttttvn

SEQ ID NO: 64 - DNA, 73 bp, Artificial Sequence
  dt_BC1004_PB
  unsure (73)..(73): /note=“v is A or C or G” /note=“n is any nucleotide”
  misc_feature (73)..(73): n is a, c, g, or t
aagcagtggtatcaacgcagagtaccacgcacacacgcgcgttttttttttttttttttttttttttttttvn

SEQ ID NO: 65 - DNA, 73 bp, Artificial Sequence
  dt_BC1005_PB
  unsure (73)..(73): /note=“v is A or C or G” /note=“n is any nucleotide”
  misc_feature (73)..(73): n is a, c, g, or t
aagcagtggtatcaacgcagagtaccactcgactctcgcgtttttttttttttttttttttttttttttttvn

SEQ ID NO: 66 - DNA, 73 bp, Artificial Sequence
  dt_BC1006_PB
  unsure (73)..(73): /note=“v is A or C or G” /note=“n is any nucleotide”
  misc_feature (73)..(73): n is a, c, g, or t
aagcagtggtatcaacgcagagtaccatatatatcagctgtttttttttttttttttttttttttttttttvn

(19) United States
(12) Patent Application Publication
(10) Pub. No.: US 2022/0291233 A1
(43) Pub. Date: Sep. 15, 2022

(54) MUCIN ISOFORMS IN DISEASES CHARACTERIZED BY BARRIER DYSFUNCTION

(71) Applicant: Universiteit Antwerpen, Antwerpen (BE)

(72) Inventors: Annemieke Smet, Sint-Niklaas (BE); Benedicte De Winter, 's-Gravenwezel (BE); Tom Breugelmans, Turnhout (BE)

(73) Assignee: Universiteit Antwerpen, Antwerpen (BE)

(21) Appl. No.: 17/627,953

(22) PCT Filed: Jun. 30, 2020
(86) PCT No.: PCT/EP2020/068340

§ 371 Date: Jan. 18, 2022

(30) Foreign Application Priority Data
Jul. 19, 2019 (EP) 19187189.6

(51) Int. Cl. G01N 33/68; C12Q 1/6883

(52) U.S. Cl. CPC G01N 33/6893; C12Q 1/6883; C12Q 2600/158; G01N 2333/4725; G01N 2800/06; G01N 2800/12

(57) ABSTRACT

The present invention relates to the field of mucin isoforms, more in particular for use in the diagnosis, monitoring, prevention and/or treatment of a disease characterized by barrier dysfunction, such as but not limited to a gastrointestinal disorder (e.g. Inflammatory Bowel Disease (IBD), Irritable Bowel Syndrome (IBS), cancer, gastro-intestinal infections, obesitas, non-alcoholic fatty liver disease (NAFLD)), neurodegenerative disorders, respiratory infections, . . . In a specific embodiment, said mucin isoform is selected from the list comprising: MUC1 isoforms and MUC13 isoforms.

### CROSS REFERENCES TO RELATED APPLICATIONS

This application is a national-stage application under 35 U.S.C. § 371 of International Application No. PCT/EP2020/068340, filed Jun. 30, 2020, which International Application claims benefit of priority to European Patent Application No. 19187189.6, filed Jul. 19, 2019.

### FIELD OF THE INVENTION

The present invention relates to the field of mucin isoforms, more in particular for use in the diagnosis, monitoring, prevention and/or treatment of a disease characterized by barrier dysfunction, such as but not limited to a gastrointestinal disorder (e.g. Inflammatory Bowel Disease (IBD), Irritable Bowel Syndrome (IBS), cancer, gastro-intestinal infections, obesitas, non-alcoholic fatty liver disease (NAFLD)), neurodegenerative disorders, respiratory infections, . . . In a specific embodiment, said mucin isoform is selected from the list comprising: MUC1 isoforms and MUC13 isoforms.

### BACKGROUND TO THE INVENTION

All epithelial tissues in the human body are covered by a mucus layer consisting of secreted and membrane-bound mucins that are a family of large molecular weight glycoproteins. Besides providing a protective function to the underlying epithelium by the formation of a physical barrier, transmembrane mucins also participate in the intracellular signal transduction. Mucins contain multiple exonic regions that encode for various functional domains. More specifically, they possess a large extracellular domain (ECD) consisting of variable number of tandem repeat (VNTR) regions rich in proline, threonine and serine (i.e. PTS domains) and heavily glycosylated. In addition, transmembrane mucins also contain extracellular epidermal growth factor (EGF)-like domains, a transmembrane region (TMD) and a shorter cytoplasmic tail (CT) that contains multiple phosphorylation sites. Binding of the ECD to the TMD is mediated by a sea urchin sperm protein, enterokinase and agrin (SEA) domain that is present in all transmembrane mucins except for MUC4. This SEA domain is autoproteolytically cleaved in the endoplasmic reticulum resulting in the noncovalent binding of the α-chain (ECD) and β-chain (TMD and CT).

Aberrant expression of transmembrane mucins has been observed during chronic inflammation and cancer. Of particular interest are MUC1 and MUC13. These transmembrane mucins are upregulated in the inflamed colonic mucosa from patients with inflammatory bowel disease (IBD) and in the tumor tissue of patients with gastric and colorectal cancer. Furthermore, emerging evidence suggests that their aberrant expression upon inflammation is associated with loss of mucosal epithelial barrier integrity.

Due to their polymorphic nature, the presence of genetic differences (i.e. single nucleotide polymorphisms (SNPs)) in mucin genes can result in different mRNA isoforms or splice variants due to alternative splicing. While most isoforms encode similar biological functions, others have the potential to alter the protein function resulting in progression toward disease. Although still poorly understood, differential expression of mucin isoforms could be involved in the pathophysiology of inflammatory diseases and cancer involving loss of barrier integrity.

Inflammatory bowel diseases (IBD), including Crohn's disease (CD) and ulcerative colitis (UC), remain disease entities with a high morbidity burden and are characterized by perpetual chronic relapsing inflammation of the intestines. At this moment, there is no curative treatment for IBD, which is why patients require life-long medication and often need surgery. Treatment mainly focuses on immunosuppression and still a substantial number of patients fail to respond or obtain full remission.

The etiology and pathogenesis of IBD are believed to involve inappropriate immune responses to the complex microbial flora in the gut in genetically predisposed persons. The intestinal mucosal barrier separates the luminal content from host tissues and plays a pivotal role in the communication between the microbial flora and the mucosal immune system. Emerging evidence suggests that loss of barrier integrity, also referred to ‘leaky gut’, is a significant contributor to the pathophysiology of IBD. The intestinal mucosal barrier comprises a thick layer of mucus, a single layer of epithelial cells and the lamina propria hosting innate and adaptive immune cells. Integrity of this barrier is maintained in several ways as depicted in FIG. 1. Secreted (e.g. MUC2) and transmembrane (e.g. MUC1, MUC4, MUC13) mucins represent the major components of the mucus barrier and are characterized by domains rich in proline, threonine, and serine that are heavily glycosylated (i.e. PTS domains). In addition to having a protective function, transmembrane mucins possess extracellular EGF-like domains and intracellular phosphorylation sites which enable them to also participate in the intracellular signal transduction. In this way, they can modulate intestinal inflammation by affecting epithelial cell proliferation, survival, differentiation and cell-cell interactions. The intestinal epithelium underneath plays an active role in innate immunity via the secretion and expression of mucins and antimicrobial peptides as well as by hosting antigen presenting cells. At this level, intense communication takes place between intestinal epithelial cells (IECs), immune cells, the microbiome and environmental antigens shaping immune responses towards tolerance or activation. IECs are mechanically tied to one another and are constantly renewed to maintain proper barrier function. This linkage is achieved by three types of intercellular junctions, listed from the apical to basal direction: tight junctions, adherens junctions and desmosomes. Whereas the adherens junctions and desmosomes are essential to maintain cell-cell adhesion by providing mechanical strength to the epithelium, tight junctions regulate paracellular permeability and seal the intestinal barrier. Tight junctions mainly consist of claudins (CLDNs), occludin (OCLN) and junctional adhesion molecules (JAMs). Apart from linking neighbouring cells, they associate with peripheral intracellular membrane proteins, such as zonula occludens (ZO) proteins, which anchor them to the actin cytoskeleton. Furthermore, tight junctions are also involved in regulating cell polarity which is established by the mutual interaction of three evolutionary conserved complexes: defective partitioning (PAR; PAR3—PAR6—aPKC), Crumbs (CRB3—PALS1—PATJ) and Scribble (SCRIB—DLG—LGL) complexes (FIG. 1). The Crumbs complex defines the apical membrane whereas the PAR and Scribble complexes are responsible for the establishment of the apical-lateral junctions between cells and the basolateral membrane, respectively. These polarity complexes are thus complementary and act together to initiate and maintain apical-basal polarity.

To date, the mechanisms underlying altered function of the intestinal mucosal barrier in IBD remain largely unexplored, particularly the role of mucins. Moehle et al., 2006 described a downregulation of MUC 2 mRNA in the colon of CD patients and increased colonic mRNA levels of MUC13 in patients with UC. This latter finding was also confirmed by another study (Sheng et al., 2011), whereas Vancamelbeke and colleagues showed a stable upregulation of MUC1 and MUC4 mRNA in both the ileum and colon of IBD patients compared to controls (Vancamelbeke et al., 2017). Upon inflammation, MUC1 and MUC13 have been shown to possess divergent actions to modulate mucosal epithelial signalling, with MUC1 being anti-inflammatory and MUC13 pro-inflammatory (Linden et al., 2008; Sheng et al., 2012). Initially, elevated MUC13 during inflammation inhibits epithelial cell apoptosis, and impairment of its expression could lower the level of protection (Sheng et al., 2011). Similarly, MUC1 protects the gastrointestinal epithelial cells from infection-induced apoptosis and enhances the rate of wound healing after injury. It should also be noted that inappropriate overexpression of transmembrane mucins could affect barrier integrity by modulating apical-basal cell polarity and cell-cell interactions, resulting in tight junction dysfunction, and may thus be responsible for the progression from local inflammation to more severe diseases, including IBD.

Therefore, in order to enhance our understanding of the role of transmembrane mucins as novel players in intestinal mucosal barrier dysfunction in IBD, we conducted an in vivo study to characterize changes in barrier components affecting integrity during the course of colitis using two complementary mouse models.

### SUMMARY OF THE INVENTION

In a first aspect, the present invention provides a mucin isoform for use in the diagnosis, monitoring, prevention and/or treatment of a disease characterized by barrier dysfunction, wherein the mucin isoform is selected from the list comprising: MUC1 isoforms and MUC13 isoforms.

In a particular embodiment, said mucin isoform is a transmembrane mucin.

In another particular embodiment, the present invention provides a mucin isoform as defined herein, for use as a biomarker for diagnosis and disease surveillance or monitoring.

In another particular embodiment, the present invention provides a mucin isoform as defined herein, for use as a new therapeutic target. In particular, said mucin isoform may be specifically targeted by monoclonal antibodies, small molecules or antisense technology.

In a specific embodiment of the present invention, said disease characterized by barrier dysfunction is a gastrointestinal disorder such as selected from the list comprising: Inflammatory Bowel Disease (IBD), Irritable Bowel Syndrome (IBS), cancer, gastro-intestinal infections, obesitas, non-alcoholic fatty liver disease (NAFLD); a neurodegenerative disorder; or a respiratory infection.

In another particular embodiment of the present invention, said cancer may be selected from the list comprising: esophageal cancer, gastric cancer, colorectal cancer, pancreas cancer, liver cancer, kidney cancer, lung cancer, ovarian cancer, colon cancer and prostate cancer.

In a further embodiment of the present invention, said gastro-intestinal infection may be selected from the list comprising: Helicobacter infection, Campylobacter infection, Clostridioides difficile infection and Salmonella infection.

In yet a further embodiment of the present invention, said neurodegenerative disorder may be selected from the list comprising: Parkinson's disease, Alzheimer's disease, Multiple Sclerosis (MS) and Autism.

In another embodiment of the present invention, said Inflammatory Bowel Disease may be selected from the list comprising: Crohn's disease and ulcerative colitis.

In yet a further embodiment, said respiratory infection may be selected from the list comprising: respiratory syncytial viral infections, influenza viral infections, rhinoviral infections, metapneumoviral infections, Pseudomonas aeruginosa viral infections and coronaviral infections. Said coronaviral infection for example being a SARS-CoV-2 infection.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1. Schematic representation of the intestinal mucosal barrier. The intestinal barrier comprises a thick layer of mucus, a single layer of epithelial cells and the inner lamina propria hosting innate and adaptive immune cells. Secreted and transmembrane mucins (MUCs) represent the major components of the mucus barrier. Besides having a protective function, transmembrane mucins also participate in intracellular signal transduction. The epithelium underneath plays an active role in innate immunity via secretion and expression of mucins and antimicrobial peptides as well as by hosting antigen presenting cells. Intestinal epithelial cells are tightly linked to each other by intercellular junctions: i.e. tight junctions (claudins (CLDNs), occludin (OCLN) and junctional adhesion molecules (JAMs)) and adherens junctions (E-cadherin and β-catenin). The PAR, Crumbs and Scribble polarity complexes regulate the polarized expression of membrane proteins in the epithelial cells.

FIG. 2. Analysis of intestinal inflammation in the adoptive T cell transfer model. (A) Schematic overview and timeline of the adoptive T cell transfer model. (B) Relative changes in body weight after T cell transfer. (C) Weekly determination of the clinical disease score by the assessment of body weight loss, pilo-erection, mobility and stool consistency. (D) Colitis severity was scored every two weeks by endoscopy and was based on the morphology of the vascular pattern, bowel wall translucency, fibrin attachment and the presence of loose stools. (E) The colon weight/length ratio. (F) At sacrifice, the colon was longitudinally opened and visually inspected for the presence of ulcerations, hyperemia, bowel wall thickening and oedema. (G) H&E stained colon sections were evaluated blinded focusing on crypt destruction, epithelial erosion, goblet cell loss and immune cell infiltration. (H) Neutrophil infiltration in the colon was assessed by measuring MPO activity. Significant differences between control and colitis mice are indicated by *p<0.05, **<0.01, ***p<0.001 (One-Way ANOVA, Tukey's multiple comparison post-hoc test).

FIG. 3. Analysis of intestinal inflammation in the DSS-induced colitis model. (A) Schematic overview and timeline of the DSS-induced colitis model. (B) Body weight was daily assessed and shown as percentage of the initial body weight. (C) Daily determination of the disease activity index (DAI), which is the cumulative score of body weight loss, the extent of rectal bleeding and changes in stool consistency. The horizontal bars indicate periods of DSS administration. (D) Rectal bleeding score. (E) The colon weight/length ratio. (F) At sacrifice, the colon was longitudinally opened and inspected for the presence of ulcerations, hyperemia, bowel wall thickening and oedema. (G) Microscopic colonic inflammation score which was based on crypt loss, epithelial erosion, goblet cell loss, immune cell infiltration and colonic hyperplasia. (H) Colonic MPO activity to assess neutrophil infiltration in the colon. N=8-14 mice/group (control, DSS cycle 1, DSS cycle 2, DSS cycle 3). Significant differences between control and colitis mice are indicated by *p<0.05, **<0.01, ***p<0.001 (One-Way ANOVA, Tukey's multiple comparison post-hoc test).

FIG. 4. Colonic cytokine expression in the T cell transfer and DSS-induced colitis models. Protein expression of pro- and anti-inflammatory cytokines in the colon of controls and T cell transfer- or DSS-induced colitis mice. Results are shown for TNF-α (A&F), IL-1β (B&G), IL-6 (C&H), IL-10 (D&I) and IL-22 (E&J). Significant differences between control and colitis mice are indicated by *p<0.05; **p<0.01; ***p<0.001 (N=5-10 mice/group (week 0 (control), 1, 2, 4 & 6) for the T cell transfer model; N=6-13 mice/group (control, DSS cycle 1, DSS cycle 2, DSS cycle 3) for the DSS model; One-Way ANOVA or Kruskal-Wallis, Tukey's and Dunn's multiple comparison post-hoc test).

FIG. 5. Analysis of intestinal permeability in the T-cell transfer and DSS-induced colitis models. Relative gastrointestinal permeability of control mice compared to colitis animals: (A) T cell transfer model (N=7-10 mice/group (week 0 (control), 1, 2, 4 & 6)); (B) DSS model (N=8-13 mice/group (control, DSS cycle 1, DSS cycle 2, DSS cycle 3)). Significant differences between control and colitis mice are indicated by *p<0.05; **p<0.01; ***p<0.001 (Kruskal-Wallis test, Dunn's post-hoc multiple comparison test).

FIG. 6. Colonic mucin expression in the adoptive T cell transfer model. (A-D) mRNA expression of Muc1, Muc2, Muc4 and Muc13 (N=7-10 mice/group (week 0 (control), 1, 2, 4 & 6)) in the colon of controls and T cell transfer-induced colitis mice. Significant differences between control and colitis mice are indicated by *p<0.05; **p<0.01; ***p<0.001 (One-Way ANOVA, Tukey's post-hoc multiple comparison test).

FIG. 7. Colonic mucin expression in the DSS-induced colitis model. (A-D) mRNA expression of Muc1, Muc2, Muc4 and Muc13 (N=10-13 mice/group (control, DSS cycle 1, DSS cycle 2, DSS cycle 3)) in the colon of controls and DSS-induced colitis mice. Significant differences between control and colitis mice are indicated by *p<0.05; **p<0.01; ***p<0.001 (One-Way ANOVA, Tukey's post-hoc multiple comparison test).

FIG. 8. Colonic intercellular junction expression in the adoptive T cell transfer model. mRNA expression of several Claudins (Cldn), Zonula-Occludens (Zo/Tjp), Junctional Adhesion Molecules (Jam), Occludin (Ocln), E-cadherin (Cdh1) and Myosin light chain kinase (Mylk) in the colon of controls and T cell transfer-induced colitis mice. Significant differences between healthy control and colitis mice is indicated by *p<0.05; **p<0.01; ***p<0.001 (N=10-13 mice/group (week 0 (control), 1, 2, 4 & 6); One-Way ANOVA or Kruskal-Wallis, Tukey's and Dunn's multiple comparison post-hoc test).

FIG. 9. Colonic intercellular junction expression in the DSS model. mRNA expression of several Claudins (Cldn), Zonula-Occludens (Zo/Tjp), Junctional Adhesion Molecules (Jam), Occludin (Ocln), E-cadherin (Cdh1) and Myosin light chain kinase (Mylk) in the colon of controls and DSS-induced colitis mice. Significant differences between control and colitis mice are indicated by *p<0.05; **p<0.01; ***p<0.001 (N=10-13 mice/group (control, DSS cycle 1, DSS cycle 2, DSS cycle 3); One-Way ANOVA or Kruskal-Wallis, Tukey's and Dunn's multiple comparison post-hoc test).

FIG. 10. Colonic expression of cell polarity proteins during the course of colitis. mRNA expression of (A) Par3, Par6, aPkcλ and aPkcζ (PAR complex) (B) Crb3, Pals1 and Patj (Crumbs complex) and (C) Scrib, Dlg1 and Llgl1 (Scribble complex) in the T cell transfer (N=7-10 mice/group (week 0 (control), 1, 2, 4 & 6)) and DSS-induced colitis model (N=10-13 mice/group (control, DSS cycle 1, DSS cycle 2, DSS cycle 3)). Significant differences between control and colitis mice are indicated by *p<0.05; **p<0.01; ***p<0.001 (One-Way ANOVA, Tukeys post-hoc multiple comparison test).

FIG. 11. Discriminant analysis with mRNA expression values of Muc1, Muc2, Muc4 and Muc13 as predictors. Discriminant analysis for the T cell transfer and DSS models to predict healthy controls and colitis groups (week 0, 1, 2, 4, 6; DSS cycle 1, DSS cycle 2, DSS cycle 3). The main predictor variables for each function are stated in the structure matrix. (A) For the T cell transfer model, the different experimental groups were mainly discriminated by Muc1 (function 1) and Muc13 (function 2). Individual mice were correctly annotated to their respective groups in 57.8% of the cases. (B) For the DSS-colitis model, the different experimental groups were primary discriminated by Muc2 (function 1) followed by Muc1 and Muc13 (function 2). Individual mice were correctly annotated to their respective groups in 69.6% of the cases.

FIG. 12. Scatter plots of correlated data for the T cell transfer model and the DSS colitis model. T cell transfer model: (A) Correlation of intestinal permeability with IL-1β protein and Muc1 mRNA expression levels. (C) Correlation of Muc1 expression with IL-1β and IL-6 protein expression. (E) Correlation of Muc1 mRNA expression with the expression levels of the intercellular junctions Cldn1 and Ocln. (G) Correlation of Muc1 mRNA expression with the expression levels of the cell polarity complex subunits Par3 and aPKCζ. DSS colitis model: (B) Correlation of intestinal permeability with TNF-α protein and Muc13 mRNA expression levels. (D) Correlation of Muc13 mRNA expression with TNF-α protein expression. (F) Correlation of Muc13 mRNA expression with the expression levels of the intercellular junctions Cldn1, Jam2 and Tjp2. (H) Correlation of Muc13 mRNA expression with the expression levels of the cell polarity complex subunits aPKCζ, Crb3 and Scrib. The correlations were selected based on the results of a multiple linear regression analysis. The corresponding adjusted R²-values and p-values of the regression model are shown.

FIG. 13. Discriminant analysis with the expression levels of cytokines, tight junctions and polarity complexes as predictors. A discriminant analysis was performed to predict healthy controls and colitis groups (weeks after T cell transfer/cycles of DSS administration) based on the expression of cytokines (protein), tight junctions (mRNA) and cell polarity proteins (mRNA) in the T cell transfer (A-C) and DSS (D-F) colitis model. The main predictor variables for each function are stated in the legend (Pooled within-groups correlations not shown). Overall, mice sacrificed 1 week after T cell transfer and after DSS cycle 1 could be clearly discriminated from control mice and the other experimental groups. (A) 72.4% of cases were correctly classified based on cytokine expression and was mainly determined by the expression of IL-1β (function 1), TNF-α and IL-6 (function 2). (B) 72.1% of cases were correctly classified based on tight junction expression and was mainly determined by the expression of Ocln (function 1) and Cldn2, Cldn1, Tjp2, Jam2 and Jam2 (function 2). (C) 84.1% of cases were correctly classified based on the expression of cell polarity proteins and was mainly determined by the expression of Par3 (function 1) and Dlg, Pat, Scrib, Llgl1 and Pals1 (function 2). (D) 37.3% of cases were correctly classified based on cytokine expression and was mainly determined by the expression of IL-1β and IL-10 (function 1) and TNF-α (function 2). In this analysis, missing values were converted to mean values, potentially explaining the bad prediction. (E) 76.5% of cases were correctly classified based on tight junction expression and was mainly determined by the expression of Jam2, Cldn2, Jam3 , Cldn15, Cldn5, Tjp1 and Cldn1 (function 1) and Tjp3, Ocln and Jam1 (function 2). (F) 64.7% of cases were correctly classified based on the expression of cell polarity subunits and was mainly determined by the expression of Par3 (function 1).

FIG. 14: Alternative mRNA transcripts of MUC1 in (a) non-inflamed and (b) inflamed colonic tissue from IBD patients. The upper panel indicates a Sashimi plot to summarize the splice junctions in the alternative mRNA transcripts. The gene structure highlighted in blue illustrates the overall exonic structure of MUC1 with the corresponding exon numbers and coding domains (CT=cytoplasmic tail; TMD=transmembrane domain; ECD=extracellular domain; EGF=epidermal growth factor; SEA=sea urchin sperm protein, enterokinase and agrin; VNTR=variable number tandem repeat; SP=signal peptide). The coloured transcripts are found in both non-inflamed and inflamed intestinal tissue. The gray mRNA transcripts highlight transcripts that are found in only one condition (i.e. inflamed or non-inflamed). On the right panel, the isoform identity number can be found of which the details are shown in table 5 (n=3 paired samples).

FIG. 15: Alternative mRNA transcripts of MUC13 in (a) non-inflamed and (b) inflamed colonic tissue from IBD patients. The upper panel indicates a Sashimi plot to summarize the splice junctions in the alternative mRNA transcripts. The gene structure highlighted in blue illustrates the overall exonic structure of MUC13 with the corresponding exon numbers and coding domains (CT=cytoplasmic tail; TMD=transmembrane domain; ECD=extracellular domain; EGF=epidermal growth factor; SEA=sea urchin sperm protein, enterokinase and agrin; VNTR=variable number tandem repeat; SP=signal peptide). The coloured transcripts are found in both non-inflamed and inflamed intestinal tissue. The gray mRNA transcripts highlight transcripts that are found in only one condition (i.e. inflamed or non-inflamed). On the right panel, the isoform identity number can be found of which the details are shown in table 5 (n=3 paired samples).

FIG. 16: RT-qPCR results to detect the SARS-CoV-2 E in the supernatants of ctrl and MUC13 siRNA transfected intestinal (LS513 and Caco2) and pulmonary (Calu3) epithelial cells infected with SARS-CoV-2 at 0.1 MOI for 48 h. Cycle threshold values are shown. Significant differences between ctrl and MUC13 siRNA transfected cells within a cell line are indicated by ### p<0.001 and between different transfected cell lines are indicated by ***p<0.001. (One-Way ANOVA, Tukey's post-hoc multiple comparison test, N=6). Error bars indicate SEM.

FIG. 17: Relative mRNA expression of ACE2 and TMPRSS2 in intestinal (LS513 and Caco2) and pulmonary (Calu3) epithelial cells infected with SARS-CoV-2 at 0.1 MOI for 24 h and 48 h. Cells treated with the growth medium of the virus were included as controls. Significant differences between SARS-CoV-2-infected and uninfected cells are indicated by *p<0.05; **p<0.01; ***p<0.001 (One-Way ANOVA, Tukey's post-hoc multiple comparison test, N=6). Error bars indicate SEM.

FIG. 18: Relative mRNA expression of the transmembrane mucins (MUC1, MUC4 and MUC13) in intestinal (LS513 and Caco2) and pulmonary (Calu3) epithelial cells infected with SARS-CoV-2 at 0.1 MOI for 24 h and 48 h. Cells treated with the growth medium of the virus were included as controls. Significant differences between SARS-CoV-2-infected and uninfected cells are indicated by *p<0.05; **p<0.01; ***p<0.001 (One-Way ANOVA, Tukey's post-hoc multiple comparison test, N=6). Error bars indicate SEM.

FIG. 19: Relative mRNA expression of the secreted mucins (MUC2, MUC5AC, MUC5B and MUC6) in intestinal (LS513 and Caco2) and pulmonary (Calu3) epithelial cells infected with SARS-CoV-2 at 0.1 MOI for 24 h and 48 h. Cells treated with the growth medium of the virus were included as controls. Significant differences between SARS-CoV-2-infected and uninfected cells are indicated by *p<0.05; **p<0.01; ***p<0.001 (One-Way ANOVA, Tukey's post-hoc multiple comparison test, N=6). Error bars indicate SEM.

FIG. 20: Relative mRNA expression of MUC13 and ACE2 in ctrl siRNA and MUC13 siRNA transfected intestinal (LS513 and Caco-2) and pulmonary (Calu3) epithelial cells infected with SARS-CoV-2 at 0.1 MOI for 48 h. Transfected cells treated with the growth medium of the virus were included as controls. Significant differences between SARS-CoV-2-infected and uninfected transfected cells are indicated by # p<0.05; ## p<0.01; ### p<0.001. Significant differences between ctrl siRNA and MUC13 siRNA transfected cells infected or uninfected with SARS-CoV-2 are indicated by ***p<0.001. One-Way ANOVA, Tukey's post-hoc multiple comparison test, N=6. Error bars indicate SEM.

FIG. 21: Relative mRNA expression of junctional proteins (CLDN1, CLDN2, CLDN3, CLDN4, CLDN7, CLDN12, CLDN15, CLDN18, OCLN, ZO-1 and ZO-2 and CHD1 (E-cadherin)) in intestinal (LS513 and Caco2) and pulmonary (Calu3) epithelial cells infected with SARS-CoV-2 at 0.1 MOI for 24 h and 48 h. Cells treated with the growth medium of the virus were included as controls. Significant differences between SARS-CoV-2-infected and uninfected cells are indicated by *p<0.05; **p<0.01; ***p<0.001 (One-Way ANOVA, Tukey's post-hoc multiple comparison test, N=6). Error bars indicate SEM.

### DETAILED DESCRIPTION OF THE INVENTION

As already detailed herein above, in a first aspect, the present invention provides a mucin isoform for use in the diagnosis, monitoring, prevention and/or treatment of a disease characterized by barrier dysfunction, wherein the mucin isoform is selected from the list comprising: MUC1 isoforms and MUC13 isoforms.

Mature mucins are composed of 2 distinct regions: the amino-and carboxy-terminal regions which are lightly glycosylated but rich in cysteines which participate in establishing disulfide linkages within and among mucin monomers; and a large central region formed of multiple tandem repeats of 10 to 80 residue sequences which are rich in serine and threonine. This area becomes saturated with hundreds of O-linked oligosaccharides.

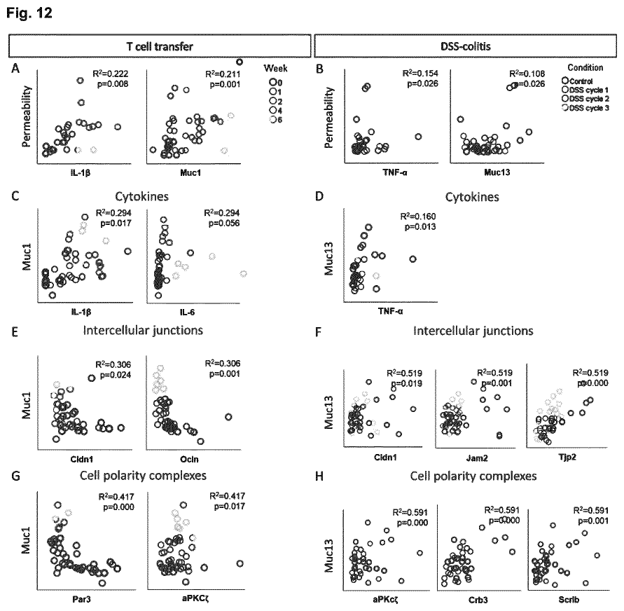

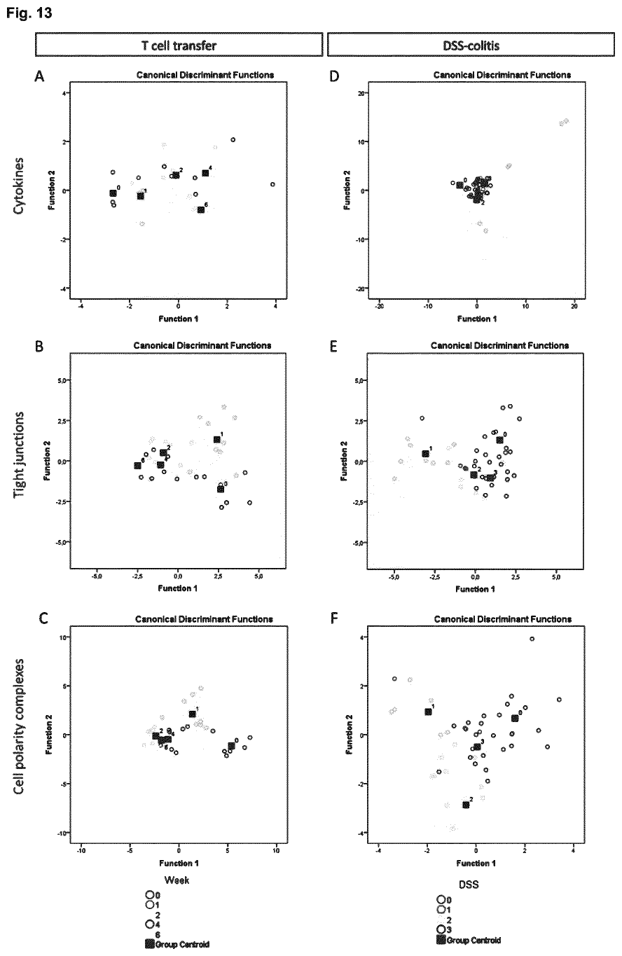

In the context of the present invention, the term “mucin isoform” is meant to be a member of a set of similar mRNA molecules or encoded proteins thereof, which originate from a single mucin gene and that are the result of genetic differences. These isoforms may be formed from alternative splicing, variable promoter usage, or other post-transcriptional modifications of the gene. Through RNA splicing mechanisms, mRNA has the ability to select different coding segments (exons) of a gene, or even different parts of exons from RNA to form different protein-mRNA sequences, i.e. isoforms. Each unique sequence produces a specific form of a protein. The presence of genetic differences in mucin genes can result in different mRNA isoforms (i.e. splice variants via alternative splicing) produced from the same mucin gene locus. While most isoforms encode similar biological functions, others have the potential to alter the protein function resulting in progression toward disease. Accordingly, the present invention is specifically directed to the identification and/or use of such mucin isoforms in various disorders. The present invention in particular provides mucin isoforms as defined herein below in the examples part, specifically those referred to in tables 5, 6, S2 and S3; as well as FIGS. 14 and 15. It further provides uses of such mucin isoforms as detailed in the present application.

The term “isoform” according to the present invention encompasses transcript variants (which are mRNA molecules) as well as the corresponding polypeptide variants (which are polypeptides) of a gene. Such transcription variants result, for example, from alternative splicing or from a shifted transcription initiation. Based on the different transcript variants, different polypeptides are generated. It is possible that different transcript variants have different translation initiation sites. A person skilled in the art will appreciate that the amount of an isoform can be measured by adequate techniques for the quantification of mRNA as far as the isoform relates to a transcript variant which is an mRNA. Examples of such techniques are polymerase chain reaction-based methods, in situ hybridization-based methods, microarray-based techniques and whole transcriptome long-read sequencing. Further, a person skilled in the art will appreciate that the amount of an isoform can be measured by adequate techniques for the quantification of polypeptides as far as the isoform relates to a polypeptide. Examples of such techniques for the quantification of polypeptides are ELISA (Enzyme-linked Immunosorbent Assay)-based, gel-based, blot-based, mass spectrometry-based, and flow cytometry-based methods.

In a particular embodiment, said mucin isoform is a transmembrane mucin, which is a type of integral membrane protein that spans the entirety of the cell membrane. These mucins form a gateway to permit/prevent the transport of specific substances across the membrane.

The specific set of disorders focused on in this application, is that they are characterized by barrier dysfunction. The term barrier dysfunction is meant to be the partial or complete disruption of the natural function of an internal barrier of a subject. Such barriers may for example include the brain barriers, the gastrointestinal mucosal barrier, the respiratory mucosal barrier, the reproductive mucosal barrier and the urinary mucosal barrier.

The gastrointestinal mucosal barrier separates the luminal content from host tissues and plays a pivotal role in the communication between the microbial flora and the mucosal immune system. Emerging evidence suggests that loss of barrier integrity, also referred to ‘leaky gut’, is a significant contributor to the pathophysiology of gastrointestinal diseases, including IBD (Inflammatory Bowel Diseases).

The blood-brain barrier is a highly selective semipermeable border of endothelial cells that prevents solutes in the circulating blood from non-selectively crossing into the extracellular fluid of the central nervous system. The blood-brain barrier restricts the passage of pathogens, the diffusion of solutes in the blood and large or hydrophilic molecules into the cerebrospinal fluid, while allowing diffusion of hydrophobic molecules (e.g. O₂, CO₂, hormones . . . ) and small polar molecules. Accordingly, an improperly functioning blood-brain barrier can be linked to neurological disorders, in particular neurodegenerative disorders. Not only the blood-brain barrier may have a role in neurological disorders, also other brain barriers, such as the blood-cerebrospinal fluid barrier, may be linked to neurological disorders.

The respiratory mucosal barrier's main function is to form a physical barrier, between the environment and the inside of an organism. It is the first barrier against continuously inhaled substances such as pathogens and allergens. Increased mucus production is often associated with respiratory infections or respiratory diseases, such as for example COPD (Chronic Obstructive Pulmonary Disease). It was moreover found that severely ill COVID-19 patients (i.e. having a SARS-CoV-2 infection) requiring intensive care, may specifically develop mucus hyperproduction in the bronchioles and alveoli of the lungs, an observation which hampers ICU stay and recovery. Accordingly, the present invention may have a significant impact on the diagnosis, monitoring, prevention and/or treatment of respiratory infections, in particular coronaviral infections such as SARS-CoV-2 infections.

Therefore, in a specific embodiment of the present invention, said disease characterized by barrier dysfunction may be a gastrointestinal disorder; a neurodegenerative disorder; cancer, or a respiratory infection.

In a particular embodiment, said gastrointestinal disorder may be selected from the list comprising: Inflammatory Bowel Disease (IBD), Irritable Bowel Syndrome (IBS), cancer, gastro-intestinal infections, obesitas, non-alcoholic fatty liver disease (NAFLD). In another embodiment of the present invention, said Inflammatory Bowel Disease may be selected from the list comprising: Crohn's disease and ulcerative colitis.

In another particular embodiment of the present invention, said cancer may be selected from the list comprising: esophageal cancer, gastric cancer, colorectal cancer, pancreas cancer, liver cancer, kidney cancer, lung cancer, ovarian cancer, colon cancer and prostate cancer.

In a further embodiment of the present invention, said gastro-intestinal infection may be selected from the list comprising: Helicobacter infection, Campylobacter infection, Clostridioides difficile infection and Salmonella infection.

In yet a further embodiment of the present invention, said neurodegenerative disorder may be selected from the list comprising: Parkinson's Disease, Alzheimer's Disease, Multiple Sclerosis (MS) and Autism.

In yet a further embodiment, said respiratory infection may be selected from the list comprising: respiratory syncytial viral infections, influenza viral infections, rhinoviral infections, metapneumoviral infections, Pseudomonas aeruginosa viral infections and coronaviral infections. Said coronaviral infection for example being a SARS-CoV-2 infection.

As used herein, the terms “treatment”, “treating”, “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “diagnosing” as used herein means assessing whether a subject suffers from a disease as disclosed herein or not. As will be understood by those skilled in the art, such an assessment is usually not intended to be correct for all (i.e. 100%) of the subjects to be identified. The term, however, requires that a statistically significant portion of subjects can be identified. The term diagnosis also refers, in some embodiments, to screening. Screening for diseases, in some embodiments, can lead to earlier diagnosis in specific cases and diagnosing the correct disease subtype can lead to adequate treatment.

In another particular embodiment, the present invention provides a mucin isoform as defined herein, for use as a biomarker for diagnosis and disease surveillance or monitoring.

By monitoring the progression and change of MUC isoform status of the individual using the methods of the present invention, the clinician or practitioner is able to make informed decisions relating to the treatment approach adopted for any one individual. For example, in certain embodiments, it may be determined that patients having specific mucin isoforms may or may not react to a particular treatment. Thus, by monitoring the response of mucin isoform carriers to various treatment approaches using the methods of the present invention, it is also possible to tailor an approach which combines two or more treatments, each targeting different subsets of isoforms in the individual.

In another particular embodiment, the present invention provides a mucin isoform as defined herein, for use as a new therapeutic target. In particular, said mucin isoform may be specifically targeted by monoclonal antibodies, small molecules or antisense technology.

### EXAMPLES

### Example 1

### Material and Methods

### Animals

Eight- to nine-week-old female immunocompromised SCID (C.B-17/Icr-Prkdc^{scid}/IcrIcoCrl) and BALB/c mice (T cell transfer model) and 7- to 8-week-old male C57BL/6J mice (DSS model) were purchased from Charles River (France). All animals were housed in a conventional animal facility with ad libitum access to food and water and a light cycle of 12 hours. After arrival in the animal facility, mice were allowed to acclimatize for 7 days before the onset of the experiments.

### Colitis Models and Experimental Design

Mouse models of colitis have been major tools in understanding the pathogenesis of IBD, yet each separate model has its limitations in that it not fully recapitulates the complexity of this human disease. Among these, the adoptive T cell transfer model has mainly been used to investigate the immunological mechanisms of intestinal inflammation mediated by T cells, and to a lesser extent to study barrier integrity. By contrast, the dextran sodium sulphate (DSS) model has been described as a useful model to examine the innate immune mechanisms involved in the development of intestinal inflammation and barrier dysfunction. More specifically, DSS is toxic to the colonic epithelium and oral administration of this chemical compound causes epithelial cell injury and innate immune responses which alter mucosal barrier integrity. As each colitis model provides valuable insights into a certain aspect of IBD, using multiple models with different initiation of pathology will thus yield a broader picture of the pathophysiology of these diseases, including barrier dysfunction.

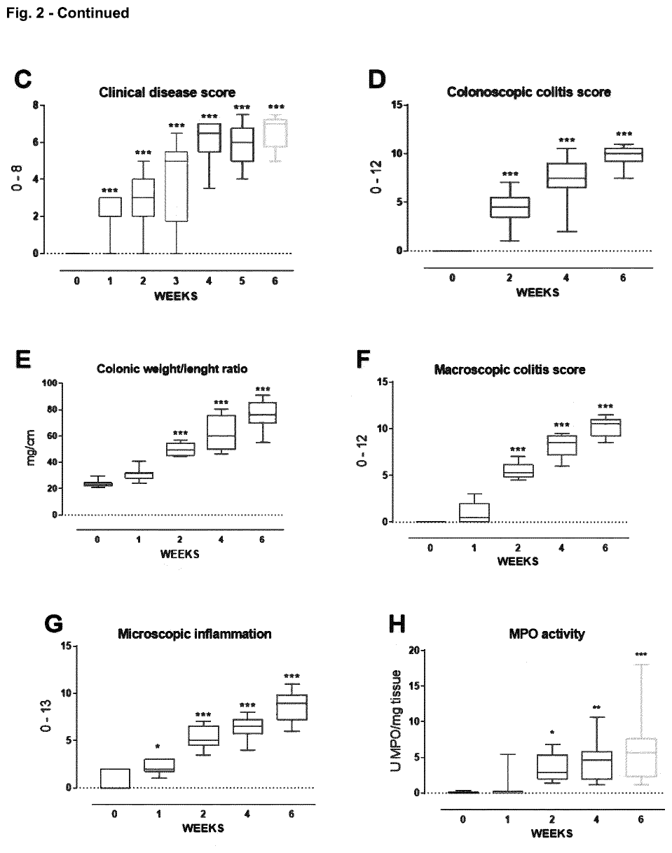

T-cell transfer model: colitis was induced in SCID mice by the adoptive transfer of CD4⁺ CD25⁻ CD62L⁺ T cells isolated from the spleens of BALB/c donor mice as described before (FIG. 2A). To monitor disease progression, animals were weighed every week and clinically scored based on the following clinical disease parameters: weight loss, piloerection, stool consistency and mobility. Each parameter was graded from 0 to 2 according to disease severity (0 =absent, 1=moderate, 2=severe; for weight loss, 0=weight gain, 1=stable, 2=weight loss). The cumulative score hence ranged from 0 to 8. In addition, intestinal inflammation was also monitored in a continuous manner in individual mice by colonoscopy at fixed time points (weeks 0, 2, 4 and 6) using a flexible Olympus URF type P5 ureteroscope with an outer diameter of 3.0 mm (Olympus Europe GmbH). Briefly, mice were sedated with a mixture of ketamine (60 mg/kg, Ketalar, Pfizer) and xylazine (6.67 mg/kg, Rompun, Bayer) (intraperitoneally (i.p.)) and placed in prone position. The anal sphincter was lubricated with gel (RMS-endoscopy) to facilitate insertion of the endoscope. Subsequently, the scope was carefully inserted through the anus as far as possible into the colon of the sedated mouse. A score was given during the withdrawal of the scope for the following parameters: morphology of the vascular pattern, bowel wall translucency, fibrin attachment and presence of loose stools (each ranging from 0 to 3), with a cumulative minimum of 0 (no inflammation) and a maximum of 12 (severe inflammation).

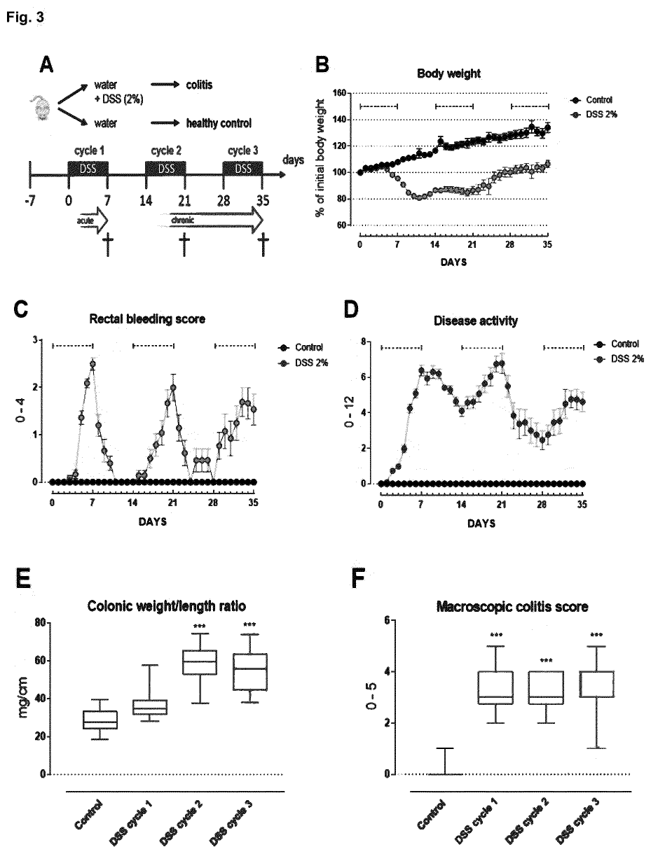

DSS-induced colitis model: acute colitis was induced by administering 2% DSS (36-50 kDa) to autoclaved drinking water for 7 days ad libitum. This cycle was repeated two more times with intermediate recovery phases of normal drinking water for 7 days to induce more chronic forms of colitis. Control mice received only autoclaved drinking water (FIG. 3A). Water levels were checked every day and were refreshed every other day. Each day, an individual disease activity index (DAI) was calculated by analysing weight loss (0=<1%, 1=1-5%, 2=5-10%, 3=10-20%, 4=>20%), stool consistency (0=normal, 1=semi-solid, 2=loose stools, 4=diarrhea) and rectal bleeding (0=no bleeding, 2=blood visible, 4=gross bleeding) to obtain a cumulative score of these parameters ranging from 0 (healthy) to 12 (severe colitis).

At 1, 2, 4 and 6 weeks post-transfer and at the end of each DSS treatment (FIGS. 2A & 3A), 10-14 animals per group (control, T cell transfer and DSS) were sacrificed by exsanguination under anesthesia (90 mg/kg ketamine and 10 mg/kg xylazine; i.p.). The collected blood was centrifuged to obtain serum for further analysis. Subsequently, the colon was resected, feces were removed and the weight as well as the length of the colon were determined and expressed as the weight/length ratio (mg/cm). Macroscopic inflammation was then scored based on the following parameters: presence of ulcerations, hyperemia, bowel wall thickening and mucosal edema. For the T cell transfer model, each parameter was scored from 0 to 3 depending on the severity, leading to a maximum cumulative score of 12 as described by Heylen et al., 2013. For the DSS model, the macroscopic scoring system of Wallace et al., 1992. was used resulting in a score from 0 to 5. Thereafter, different samples from the colon (distal side) were taken and processed immediately or stored in RNA later, snapfrozen or embedded in paraffin or cryoprotectant until further analysis (see below).

### Myeloperoxidase (MPO) Activity Assay

Myeloperoxidase (MPO) activity was measured in colonic tissue as a parameter for neutrophil infiltration (Heylen et al., 2013). Briefly, colonic samples were immersed in potassium phosphate (pH 6.0) containing 0.5% hexadecyltrimethylammonium bromide (0.02 mL/mg tissue). Thereafter, samples were homogenized, subjected to two freeze-thawing cycles and subsequently centrifuged at 15000 rpm for 15 min at 4° C. An aliquot (0.1 mL) of the supernatant was then added to 2.9 mL of o-dianisidine solution (i.e. 16.7 mg of o-dianosidine dihydrochloride in 1 mL of methyl alcohol, 98 mL of 50 mM potassium phosphate buffer at pH 6.0 and 1 mL of 0.005% H₂O₂ solution). Immediately afterwards, the change in absorbance of the samples was read at 460 nm over 60 sec using a Spectronic Genesys 5 spectrophotometer (Milton Roy). One unit of MPO activity equals the amount of enzyme able to convert 1 mmol of H₂O₂ to H₂O per min at 25° C.

### RNA Extraction and RT-qPCR for Gene Expression

Total RNA from colonic tissue stored in RNA later, was extracted using the NucleoSpin® RNA plus kit (Macherey-Nagel) following the manufacturer's instructions. The concentration and quality of the RNA were evaluated using the NanoDrop® ND-1000 UV-Vis Spectrophotometer (Thermo Fisher Scientific). Subsequently, 1 μg RNA was converted to cDNA by reverse transcription using the SensiFast™ cDNA synthesis kit (Bioline). Relative gene expression was then determined by SYBR Green RT-qPCR using the GoTaq qPCR master mix (Promega) on a QuantStudio 3 Real-Time PCR instrument (Thermo Fisher Scientific). Primer sequences are shown in Supplementary Table 1.

Supplementary table Si.
Primer sequences used in qPCR assays

| Gene name | Primer | SEQ ID No | Primer sequence (5′-3′) |
|---|---|---|---|
| Cdh1 | FW | 1 | CAGTTCCGAGGTCTACACCTT |
| | REV | 2 | TGAATCGGGAGTCTTCCGAAAA |
| Cldn1 | FW | 3 | TGCCCCAGTGGAAGATTTACT |
| | REV | 4 | CTTTGCGAAACGCAGGACAT |
| Cldn2 | FW | 5 | CAACTGGTGGGCTACATCCTA |
| | REV | 6 | CCCTTGGAAAAGCCAACCC |
| Cldn3 | FW | 7 | ACCAACTGCGTACAAGACGAG |
| | REV | 8 | CGGGCACCAACGGGTTATAG |
| Cldn5 | FW | 9 | GCAAGGTGTATGAATCTGT |
| | REV | 10 | GTCAAGGTAACAAAGAGTGCCA |
| Cldn7 | FW | 11 | GGCCTGATAGCGAGCACTG |
| | REV | 12 | TGGCGACAAACATGGCTAAGA |
| Cldn15 | FW | 13 | ATTGCAGGGACCCTCCACATA |
| | REV | 14 | GCCCAGTTCATACTTGGTTCC |
| Crb3 | FW | 15 | CACCGGACCCTTTCACAAATA |
| | REV | 16 | CCCACTGCTATAAGGAGGACT |
| Dlg1 | FW | 17 | AGTGACGAAGTCGGAGTGATT |
| | REV | 18 | GTCAGGGATCTCCCCTTTATCT |
| Jam1 | FW | 19 | TCTCTTCACGTCTATGATCCTGG |
| | REV | 20 | TTTGATGGACTCGTTCTGGGG |
| Jam2 | FW | 21 | GTGCCCACTTCTGTTATGACTG |
| | REV | 22 | TTCCCTAGCAAACTTGTGCCA |
| Jam3 | FW | 23 | CTGCGACTTCGACTGTACG |
| | REV | 24 | TTCGGTTGCTGGATTTGAGATT |
| Llgl1 | FW | 25 | GCTTCCCCAATCAGCCCAG |
| | REV | 26 | GCGCAGCCATTATGATGGATG |
| Muc1 | FW | 27 | GGTTGCTTTGGCTATCGTCTATTT |
| | REV | 28 | AAAGATGTCCAGCTGCCCATA |
| Muc2 | FW | 29 | ATGCCCACCTCCTCAAAGAC |
| | REV | 30 | GTAGTTTCCGTTGGAACAGTGAA |
| Muc4 | FW | 31 | ACAGGTGTAACTAGAAGCCTCG |
| | REV | 32 | CAGGGGTGCTATGCACTACTG |
| Muc13 | FW | 33 | GCCAGTCCTCCCACCACGGTA |
| | REV | 34 | CTGGGACCTGTGCTTCCACCG |
| Mylk | FW | 35 | TGGGGGACGTGAAACTGTTTG |
| | REV | 36 | GGGGCAGAATGAAAGCTGG |
| Ocln | FW | 37 | GGCGGATATACAGACCCAAGAG |
| | REV | 38 | GATAATCATGAACCCCAGGACAAT |
| Pals1 | FW | 39 | TTTGGGCACCAGAATGATGC |
| | REV | 40 | AACAATTCCTTCTTCCGTGTCAA |
| Pat3 | FW | 41 | GGAGATGGCCGCATGAAAGTT |
| | REV | 42 | CTCCAAGCGATGCACCTGTAT |
| Pat6 | FW | 43 | TCAGAAACGGGCAGAAGGTG |
| | REV | 44 | CCAGGCGGGAGATGAAGATA |
| Patj | FW | 45 | TTCGATGGGCACCACTATATC |
| | REV | 46 | GGTGGGGGCACTTCTTTAAGG |
| aPkcλ | FW | 47 | CACTTTGAGCCTTCCATCTCC |
| | REV | 48 | GTGACCAGCTTGTGGCACT |
| aPkcζ | FW | 49 | GCGTGGATGCCATGACAACAT |
| | REV | 50 | GGCTCTTGGGAAGGCATGACA |
| Rp14 | FW | 51 | CCGTCCCCTCATATCGGTGTA |
| | REV | 52 | GCATAGGGCTGTCTGTTGTTTTT |
| Scrib | FW | 53 | CCTGGGCATCAGTATCGCAG |
| | REV | 54 | GCCCTCGTCATCTCCTTTGT |
| Tjp1 | FW | 55 | GAGCGGGCTACCTTACTGAAC |
| | REV | 56 | GTCATCTCTTTCCGAGGCATTAG |
| Tjp2 | FW | 57 | ATGGGAGCAGTACACCGTGA |
| | REV | 58 | TGACCACCCTGTCATTTTCTTG |
| Tjp3 | FW | 59 | CTGTGGAGAACGTCACATCTG |
| | REV | 60 | CGGGGACGCTTCACTGTAAC |

All RT-qPCR reactions were performed in duplicate and involved an initial DNA polymerase activation step for 2 min at 95° C., followed by 40 cycles of denaturation at 95° C. for 15 sec and annealing/extension for 1 min at 60° C. Analysis and quality control were performed using qbase+software (Biogazelle). Relative expression of the target genes was normalized to the expression of the housekeeping genes Actb and Rpl4.

### Quantification of Intestinal Permeability

To assess in vivo intestinal permeability, the FITC-dextran intestinal permeability assay was performed as described by Gupta et al., 2014. In brief, mice were intragastrically inoculated 4 hours prior to euthanasia with FITC-dextran (44 mg/100 g body weight (T cell transfer), 60 mg/100 g body weight (DSS model), 4 kDa, Sigma). Upon euthanasia, blood was collected via cardiac puncture and transferred into SSTII Advance Blood Collection Tubes (BD Vacutainer). After centrifugation (10000 rpm, 5 min), serum was collected and equally diluted with PBS. Subsequently, aliquots of 100 μl were added in duplo to a 96-well microplate and the concentration of FITC was measured by spectrophotofluorometry (Fluoroskan Microplate Fluorometer, Thermo Fisher Scientific) at an excitation wavelength of 480 nm and an emission wavelength of 530 nm. The exact FITC-dextran concentration per well was calculated using a standard curve with serially diluted FITC-dextran solutions.

### Cytokine Measurements

To determine colonic inflammatory mediators at protein level, two different approaches were applied. First, fresh colonic segments were rinsed with PBS, blotted dry and weighed. Subsequently, the samples were stored on ice until further processing in a Tris-EDTA buffer (i.e. PBS containing 10 mM Tris, 1 mM EDTA, 0.5% v/v Tween-20 and a protease-inhibitor cocktail (Sigma-Aldrich)) at a ratio of 100 mg tissue per ml buffer. Samples were then homogenized, centrifuged (11 000 rpm, 10 min, 4° C.) and the supernatants were stored at −80° C. until further analysis. Colonic cytokine levels were quantified using cytometric bead arrays (CBA) (BD Biosciences) for Tumour Necrosis Factor (TNF)-α, Interferon (IFN)-γ, Interleukin (IL)-1β and IL-6 according to the manufacturer's instructions. Fluorescence detection was performed on a BD Accuri C6 flow cytometer and the FCAP Array software was used for data analysis.

Second, snap frozen colonic tissues were homogenized using beads and total protein was extracted in ice cold NP-40 buffer (i.e. 20 mM Tris HCl (pH 8), 137 mM NaCl, 10% glycerol, 1% nonidet-40, 2 mM EDTA) supplemented with protease and phosphatase inhibitor cocktail tablets (Roche). After centrifugation (14.000 rpm, 10 min, 4° C.) to remove cell debris, the protein concentration was determined using the Pierce BCA protein assay kit (Thermo Fisher Scientific). Enzyme-Linked ImmunoSorbent Assay (ELISA) was then performed to quantify colonic cytokine expression at the protein level. To this end, the mouse uncoated ELISA kits (Invitrogen) were used according to the manufacturer's instructions to measure protein concentrations of IL-1β, TNF-α, IL-6, IL-10 and IL-22. A standard curve was created by performing 2-fold serial dilutions of the top standards included in the kits. For each sample, 100 μl of a 2.5 μg/ml protein solution was analysed by ELISA in duplicate.

### Histopathology and Immunohistochemistry

In order to evaluate inflammation at the microscopic level, full thickness colonic segments were fixed for 24 h in 4% formaldehyde and subsequently embedded in paraffin. Cross sections (5 μm thick) were deparaffinized and rehydrated. Sections were then stained with Hematoxylin Gill III Prosan (Merck) and Eosin Yellow (VWR) according to the standardized protocols. Inflammation was scored based on the degree of inflammatory infiltrates (0-3), presence of goblet cells (0-1), crypt architecture (0-3), mucosal erosion and/or ulceration (0-2), presence of crypt abscesses (0-1) and the number of layers affected (0-3), resulting in a cumulative score ranging from 0 to 13 (Moreels et al., 2004). Periodic Acid-Schiff (PAS) staining was performed to detect mucin glycoproteins in paraffin-embedded colon sections. In brief, rehydrated 5 μm thick colon sections were placed in Schiff reagent for 15 min after an initial oxidation step in 0.5% periodic acid solution for 5 min. Then, colon sections were washed with tap water, counterstained with hematoxylin and analysed by light microscopy (Olympus BX43).

Several immunohistochemical mucin stainings were also applied on paraffin-embedded colonic tissue using the following primary antibodies: the polyclonal rabbit Muc1 (Abcam (ab15481), 1/1000), Muc2 (Novus Biologicals (NBP1-31231), 1/3000), Muc4 (Novus Biologicals (NBP1-52193SS), 1/3000) and the in-house Muc13 ( 1/2000) antibodies. Briefly, heat-induced antigen retrieval was performed in EDTA (pH 8) (MUC1 and MUC13) or citrate buffer (10 mM, pH 6) (MUC2 and MUC4). Subsequently, endogenous peroxidase activity was blocked by incubating the slides with 3% H₂O₂ in methanol (5 min). Primary antibody incubation was performed overnight at 4° C. Subsequently, the mucins were visualized by incubating the colon sections with a goat anti-rabbit biotinylated secondary antibody (EnVision detection system for MUC13) for 60 min at room temperature, followed by incubation with HRP-avidin complexes. Finally, visualization of the target antigen was performed by a short incubation with aminoethyl carbazole (AEC), after which the sections were counterstained with hematoxylin. Washing steps were performed using Tris-buffered saline containing 0.1% Triton X-100 (pH 7.6). The stainings were analysed by light microscopy (Olympus BX43).

To visualize tight junctions in the colon, fresh colonic tissue was transversally placed and immersed in Richard-Allan Scientific™ Neg50™ Frozen Section Medium (Thermo Fisher Scientific) and snap frozen, after which 6μm cryosections were mounted on SuperFrost slides (Thermo Fisher Scientific). After a short fixation period of 5 min in aceton, the sections were dried and rinsed with Tris-buffered saline supplemented with 1% albumin. The sections were then incubated overnight with the following primary antibodies: ZO-1 (Invitrogen (61-7300), 1/1000) and CLDN1 (abcam (ab15098), 1/2000). The next day, secondary antibody incubation was performed for 60 min using a goat anti-rabbit Alexa Fluor 594 secondary antibody (Invitrogen, 1/800). After rinsing in distilled water, the colon sections were counterstained and protected against fading using Vectashield mounting medium containing DAPI (Vector Laboratories). Washing steps were performed using Tris-buffered saline supplemented with 0.1% Triton X-100. For visualization, a Nikon Eclipse Ti inverted fluorescence microscope equipped with a Nikon DS-Qi2 camera was used. All sections were blinded to obtain the representative images.

### Statistics

Statistical analysis using the GraphPad Prism 8.00 software (licence DFG170003) was performed to determine significant differences between control and the different colitis groups within a certain model (T cell transfer or DSS). Data were analysed by the One-way Analysis of Variance (ANOVA) and non-parametric Kruskal-Wallis tests and are presented as means±standard error of mean (SEM) or boxplots (min to max), unless stated otherwise. Significance levels are indicated on the graphs by *p<0.05, **<0.01, ***p<0.001 and were corrected for multiple testing using the Tukey-Kramer's and Dunn's post-hoc multiple comparisons tests.

A discriminant function analysis was performed to determine whether colitis mice could be distinguished from control animals based on a set of predictor variables (i.e. the expression of cytokines, mucins or other barrier mediators). The results are depicted as scatter plots showing the two main discriminant functions (i.e. function 1 and function 2) with the according main predictor variables summarized in a table. Furthermore, a multiple linear regression analysis was carried out to investigate associations (1) between changes in barrier integrity and the expression of mucins, cytokines and barrier mediators; (2) between the expression of mucins, cytokines and barrier mediators. Scatter plots are shown distinguishing between different experimental groups with the corresponding p-value of the regression model. A p-value below 0.05 was considered statistically significant. These analyses were performed using IBM SPSS Statistics 24 software.

### Results

### Macroscopic and Microscopic Observations of Colitis Evolution Over Time

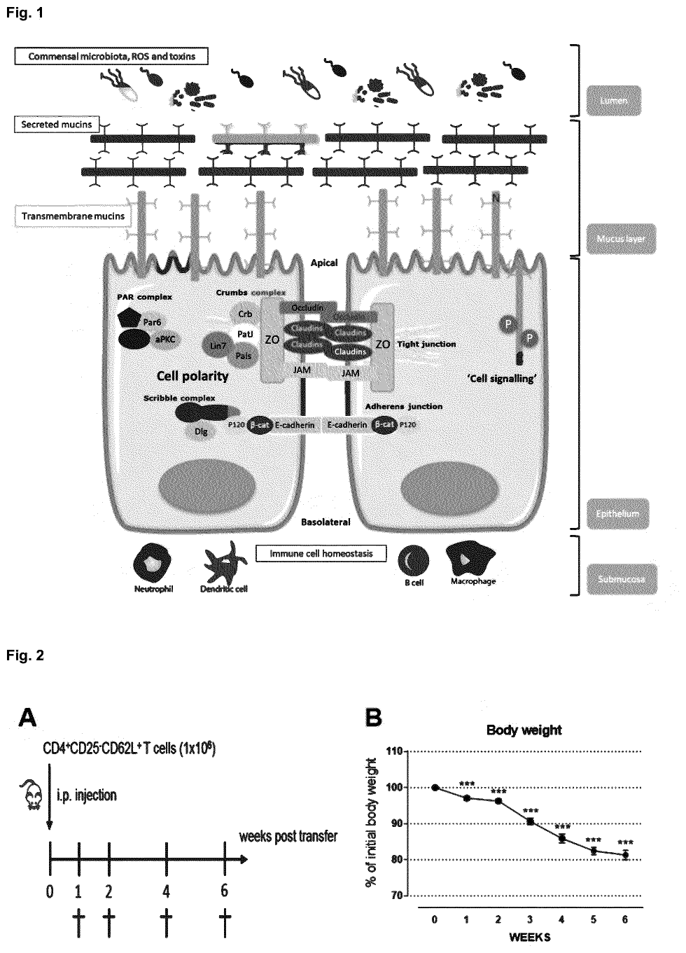

In the T cell transfer model, SCID mice started to develop clinical signs of colitis one week after the adoptive transfer of naive T cells. Body weight was decreased at 1 week post-transfer compared to the initial body weight pre-transfer and this decrease gradually continued until week 6 (FIG. 2B). The clinical disease score increased over time starting from week 1 to week 4, while stagnating afterwards (FIG. 2C). A colonoscopy was performed every 2 weeks to monitor signs of colitis in the bowel wall, showing a time-dependent increase in inflammatory scores at weeks 2, 4 and 6 post-transfer compared to the control mice (FIG. 1D). After sacrifice, the mucosal damage in the colon was scored at both a macro- and microscopic level. Mice that were sacrificed at 2, 4- and 6-weeks post-transfer showed a gradual increase in macroscopic inflammation (FIG. 2F). This phenomenon was also seen for another macroscopic marker of colonic inflammation, the colonic weight/length ratio, which is a quantification of colonic edema (FIG. 2E). In contrast, the infiltration of neutrophils and lymphocytes became already visible on H&E-stained colonic segments of colitis mice at week 1 post-transfer (FIG. 2G). This mucosal and submucosal infiltration of immune cells gradually increased and was associated with a remarkable increase in colon thickness as disease progressed to weeks 2, 4 and 6 (FIG. 2G). Furthermore, MPO activity, which is caused by neutrophil infiltration in the mucosa, was increased starting from 2 weeks post-transfer, with a gradual increase over time to weeks 4 and 6 (FIG. 2H).

In the DSS colitis model, mice treated with DSS started to lose weight after 5 days of DSS administration in the first cycle. The body weight further decreased when normal drinking water was reintroduced at day 8, with a maximal weight loss at day 11 of the experimental protocol (FIG. 3B). The colitis mice started to regain weight at the end of the second DSS cycle (day 21) until the initial body weight was reached at the end of the experiment. Healthy control mice gained weight over time (FIG. 3B). As a result of DSS administration, mice in each DSS group showed maximal changes in stool consistency and rectal bleeding after 7 days of DSS administration, which decreased and completely disappeared in the recovery phase (FIG. 3). The above-described parameters to assess clinical disease in this model (body weight, stool consistency and rectal bleeding) are combined in the DAI score, which is shown in FIG. 3D. Control mice did not show any signs of disease throughout the experiment, whereas administration of 2% DSS for 7 days stably induced a mild acute colitis after DSS cycle 1. The two subsequent DSS cycles, however, led to the development of a chronic colitis with an increased interindividual variability.

To assess the effect of DSS-induced colitis on macro- and microscopic inflammatory parameters of the colon, a group of mice was sacrificed after each cycle of DSS administration (DSS cycle 1, DSS cycle 2 and DSS cycle 3, respectively, FIG. 3A). The colonic weight/length ratio was increased in all three groups (cycles 1, 2 and 3) compared to the control group. The macroscopic inflammation score was increased in all DSS cycles (FIG. 3F) with hyperemia and ulcerations abundantly present after DSS cycle 1, whereas colon thickening appeared after DSS cycles 2 and 3. Microscopic inflammation was present in all DSS groups as scored on H&E-stained colon sections (FIG. 3G) and showed crypt loss, epithelial erosions and marked infiltration of neutrophils in the colon of acute DSS treated mice (data not shown). At the end of DSS cycles 2 and 3, the colon sections showed epithelial regeneration compared to the acute stage, yet with remarkable hyperplasia. Infiltration of neutrophils and lymphocytes in the submucosa and mucosa could also be observed (data not shown). In addition, some mice even showed massive focal ulcerations in the colon. At the molecular level, MPO activity was increased during DSS-induced colitis progression (FIG. 3H), which confirmed the infiltration of neutrophils into the colon due to DSS administration. Interestingly, mice treated with 3 DSS cycles showed a significant lower colonic MPO activity compared to mice treated only once.

### Colonic Inflammatory Markers

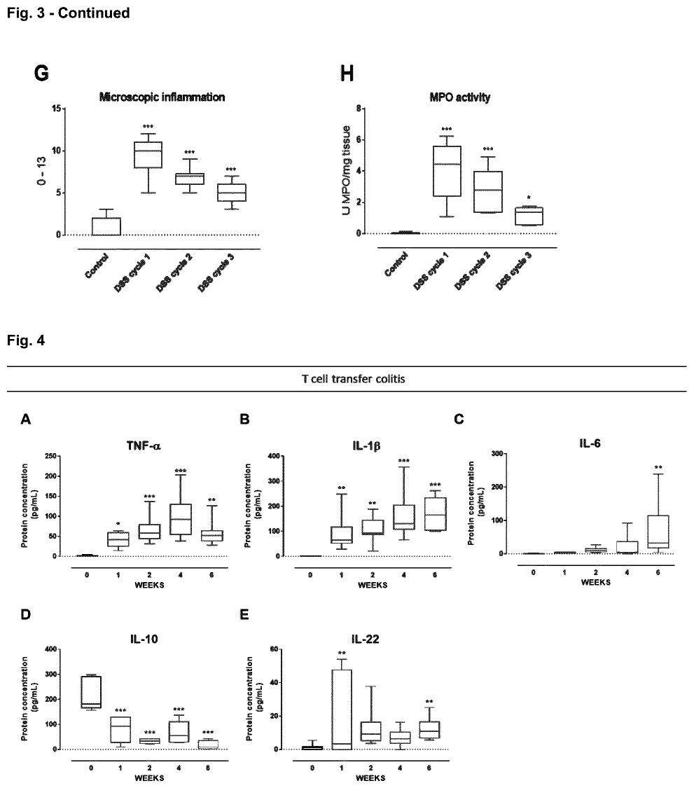

In both colitis models, colonic protein levels of several inflammatory markers were quantified as shown in FIG. 4. At all timepoints post-transfer and after each cycle of DSS administration, expression of IL-β and TNF-α was increased whereas IL-10 was reduced in expression (FIG. 4A-B, D, F-G, I). Interestingly, IL-22 protein levels were only increased at 1 and 6 weeks post-transfer and at the end of DSS cycles 1 and 3 (FIG. 4E, J). In contrast, expression of IL-6 was only increased in the more chronic phase of colitis, i.e. at week 6 post-transfer (FIG. 4C) and after the second cycle of DSS administration (FIG. 4H).

### Mucosal Barrier Function During Colitis Progression

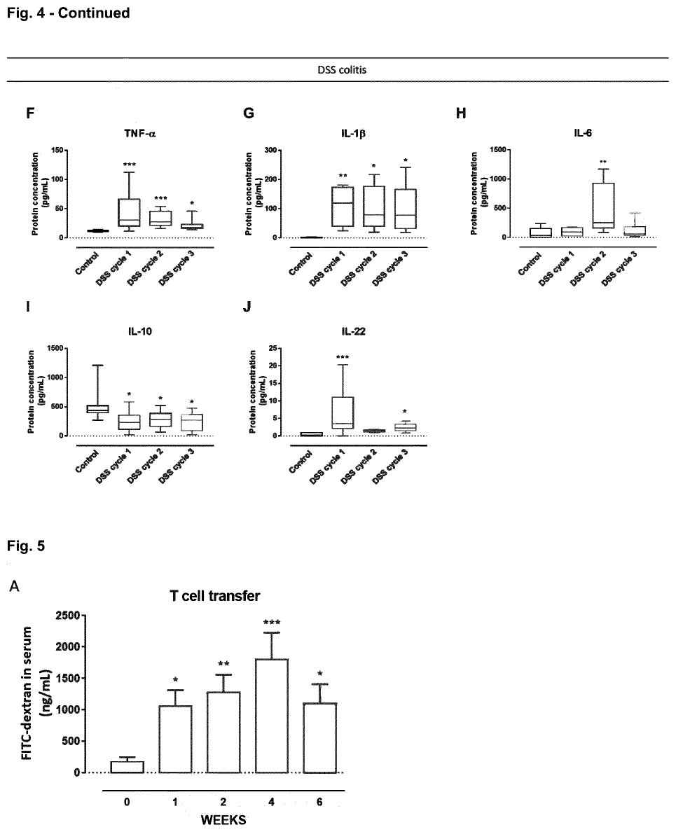

As loss of intestinal barrier integrity is recognized as a major hallmark of the IBD pathophysiologyl⁸, changes in barrier permeability during colitis progression were investigated in both models. Results of the FITC-dextran intestinal permeability assays showed that integrity of the intestinal mucosal barrier was affected in both models (FIG. 5). More specifically, intestinal permeability progressively increased during colitis progression in the T cell transfer model levelling off at week 6, but remaining increased as compared to control mice (FIG. 5A). In the DSS model, intestinal permeability showed a strong increase after the first cycle of DSS administration, after which it declined in the chronic stages of colitis with only a significant increase left after the second DSS cycle but not after the third cycle (FIG. 5B).

To further substantiate intestinal mucosal barrier dysfunction upon colitis, the expression of several components that are the building stones of and regulate the mucosal barrier were measured.

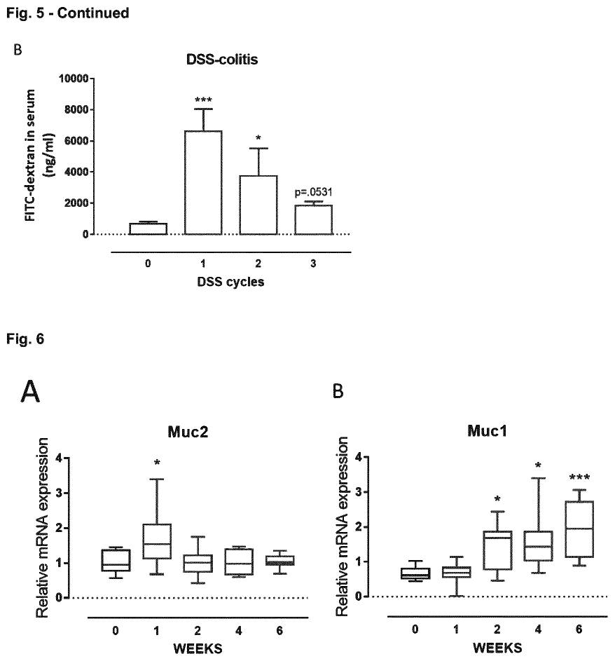

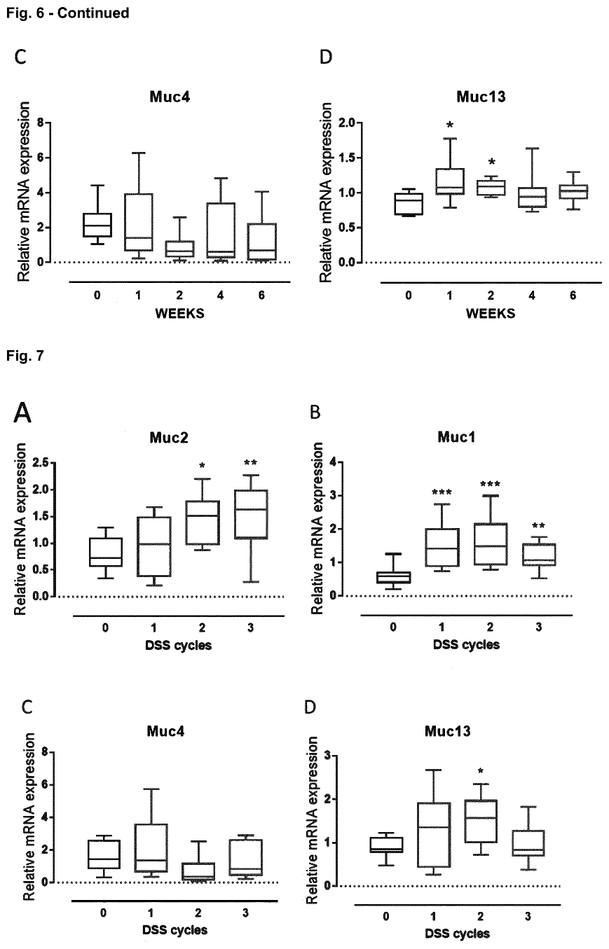

We first investigated mucin expression since mucins constitute the main part of the mucus layer and are the first barrier luminal pathogens and toxins encounter. Muc2 (i.e. the main secreted mucin of the large intestine) mRNA expression was increased after 1 week post-transfer (FIG. 6A) whereas it was upregulated during the chronic stages of DSS-induced colitis (FIG. 7A). mRNA expression of Muc1, a transmembrane mucin expressed only at low levels in the healthy intestines, was upregulated after 2, 4 and 6 weeks post-transfer (FIG. 6B) and after all cycles of DSS administration (FIG. 7B). The transmembrane Muc13 mucin, which is normally expressed in the healthy intestines, showed aberrant expression patterns at the RNA level in both models with an increased expression seen at 1 and 2 weeks after T cell transfer and DSS cycle 2 (FIGS. 6D & 7D). In contrast, mRNA expression of Muc4, another membrane-bound mucin, was not significantly altered during experimental colitis in either model (FIGS. 6C & 7C). The changes in mucin mRNA expression were verified at protein level by immunohistochemical stainings (data not shown). In the DSS model, we observed increased Muc2 staining intensity during colitis progression, whereas in the T cell transfer model, overall Muc2 staining intensities were not altered compared to control animals. In control animals, Muc1 was mainly observed on the apical side of epithelial cells lining the villi, whereas colitis induction was associated with increased Muc1 staining intensities in the cytoplasm and the crypts in both colitis models. Muc13 intensity was mainly increased after the first two cycles of DSS administration and from week 2 post-transfer in the T cell transfer model. Concerning its cellular localisation, Muc13 showed a strong apical staining intensity in intestinal epithelial cells, which became apparent in the cytoplasm during colitis. For Muc4, no clear changes were observed during colitis progression compared to control animals.

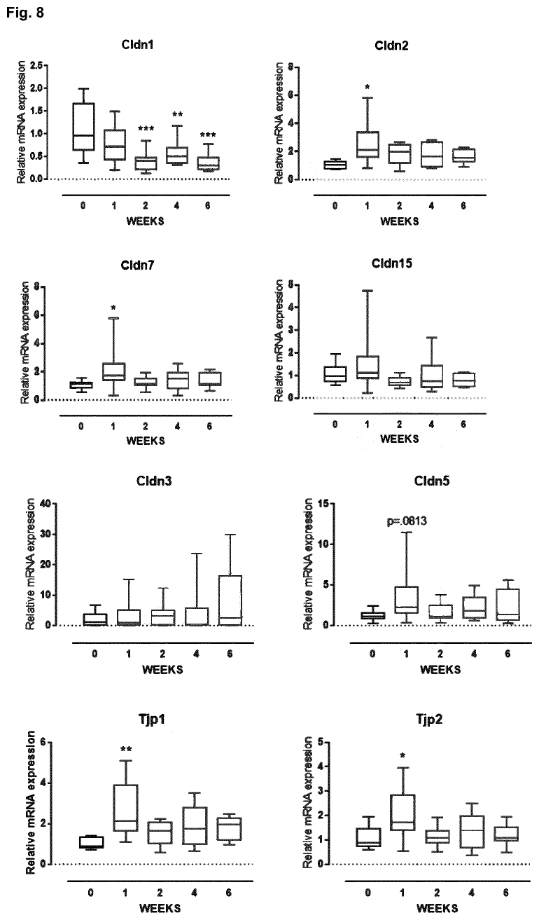

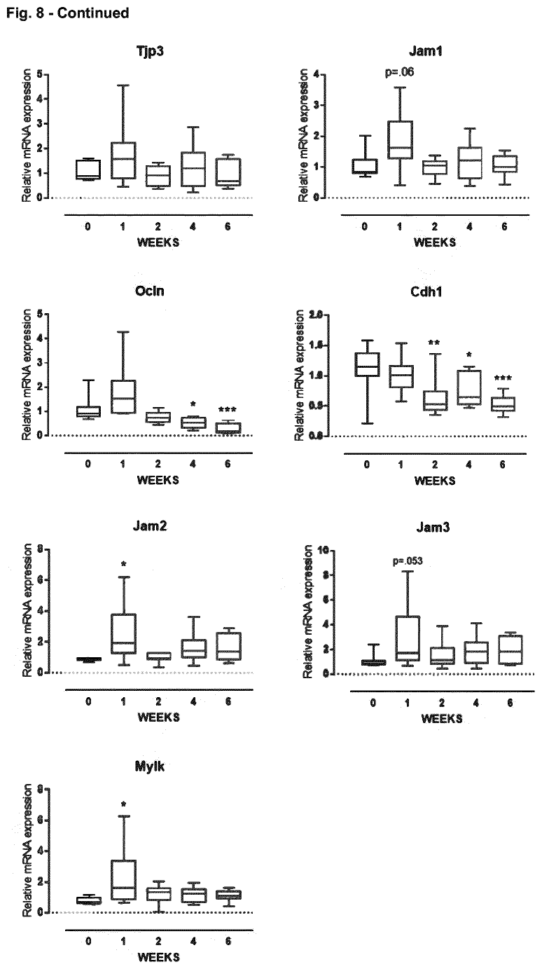

Several interesting alterations were observed in both models as far as the expression patterns of junction constituents at RNA level were concerned (FIGS. 8 & 9). mRNA expression levels of Zo1 (Tjp1), Tjp2, Jam2, Jam3 and Myosin Light Chain Kinase (Mylk) were significantly increased at week 1 post-transfer and after the first cycle of DSS administration (FIGS. 8 & 9). E-cadherin (Cdh1) and Ocln mRNA expression levels were significantly decreased during the more chronic stages of experimental colitis in both models (FIGS. 8 & 9). mRNA levels of Cldn1, a major regulator of paracellular permeability, were elevated after the first DSS cycle, whereas it decreased throughout colitis progression in the T cell transfer model (FIGS. 8 & 9). In contrast, Cldn2 mRNA expression was increased at 1 week post-transfer, yet its expression declined at the end of each DSS cycle (FIGS. 8 & 9). In addition, Cldn5 and Cldn7 showed a model-specific response. More specifically, expression of Cldn7 and Cldn5 mRNA was upregulated at the initial stage of colitis in the T cell transfer and the DSS model, respectively (FIGS. 8 & 9). Furthermore, Tjp3 mRNA expression was reduced throughout colitis progression in the DSS-induced colitis model only, whereas Cldn15 mRNA expression was significantly decreased during the acute phase of DSS-induced colitis and became significantly increased in the chronic phases (FIG. 9). Expression of Cldn3 and Jam1 was not altered throughout colitis progression in either model (FIGS. 8 & 9). Immunohistochemical stainings for ZO-1 and CLDN1 were also performed to analyse alterations in intercellular junctions at the protein level. These results showed that mainly CLDN1 showed an increased staining intensity during the course of colitis in both models highlighting dysfunction of this tight junction protein, whereas no clear alterations could visually be observed for ZO-1 (data not shown).

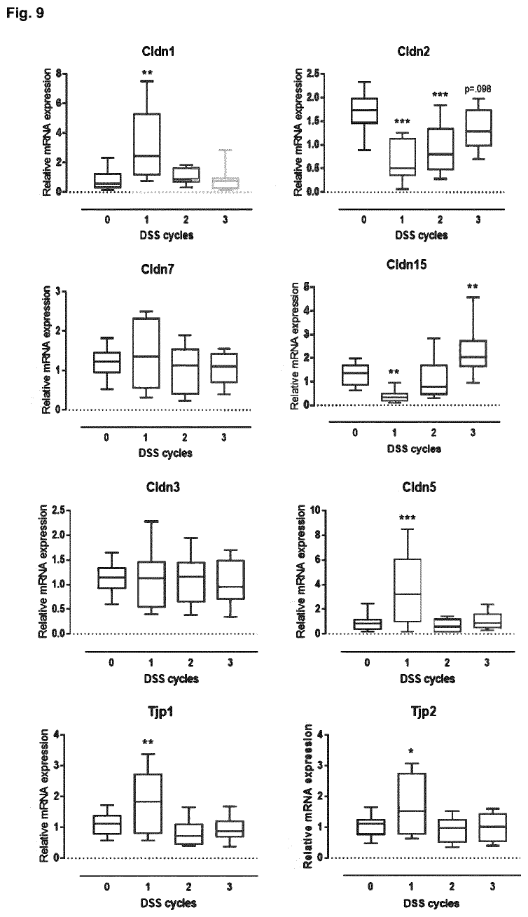

In addition to appropriate expression of intercellular junctions, a well organised apical-to-basal cell polarity is indispensable for the formation of a functional and tight intestinal epithelial cell monolayer. Gene expression analysis showed that subunits of the different polarity complexes were affected in both our experimental colitis mouse models (FIG. 10). The expression of Par3 and aPkcλ, two major coordinators of tight junction localization, was downregulated at all DSS cycles and time points post-transfer (FIG. 10A). On the other hand, aPkcζ mRNA expression was only decreased in the T cell transfer model, whereas Par6 mRNA expression was only elevated at the acute phase of DSS-induced colitis (FIG. 10A). Regarding the subunits of the Crumbs polarity complex as shown in FIG. 10B, Patj mRNA expression tended to be decreased at all DSS cycles, whereas its expression was upregulated at week 1 post-transfer. Also mRNA expression of Pals1 (Mpp5) was upregulated at the first time-point of the T cell transfer model (FIG. 10B). No significant alterations in Crb3 expression were observed in either colitis models (FIG. 10B). Interestingly, Scrib expression, which is known to be a negative regulator of the PAR complex, was increased at 1 week post-transfer and after the first DSS cycle (FIG. 10C). Although expression of Dlg1 and Llgl1 was altered in the T cell transfer model at 1 and 2 weeks post-transfer, respectively, no changes in expression of these subunits were observed in the DSS-colitis model (FIG. 10C). The above results highlight that epithelial cell polarity is disturbed as a consequence of colitis induction, both in the acute and chronic stages.

### Aberrant Mucin Expression Associated With Loss of Barrier Integrity Upon Inflammation

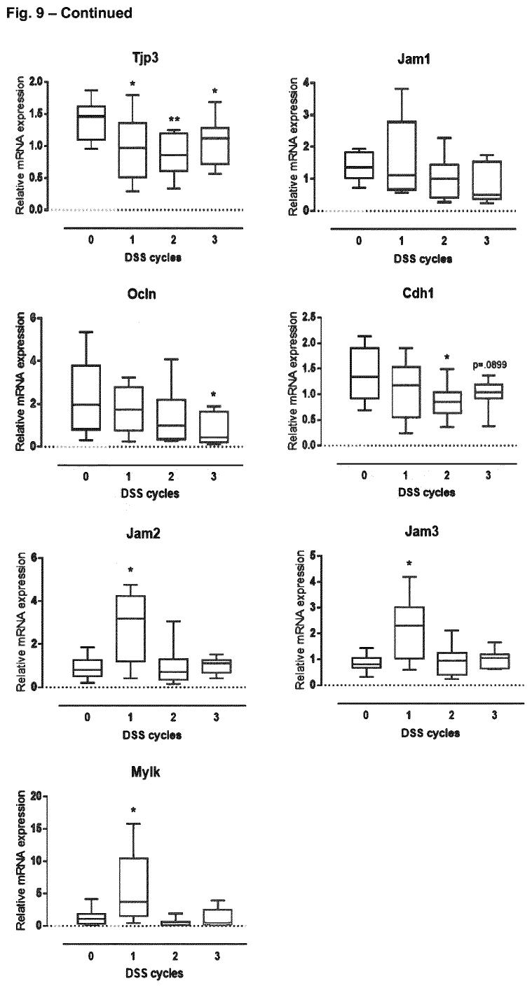

It has been suggested that overexpression of transmembrane mucins in many cancer types can contribute to loss of epithelial barrier integrity by mediating junctional and cell polarity dysfunction. To elucidate the involvement of aberrantly expressed transmembrane mucins as potential mediators in intestinal mucosal barrier disruption upon inflammation-induced colitis, the mucin mRNA expression data were used to perform a discriminant analysis on both models and to correlate the changes in intestinal permeability and colonic inflammation (FIGS. 11 & 12).

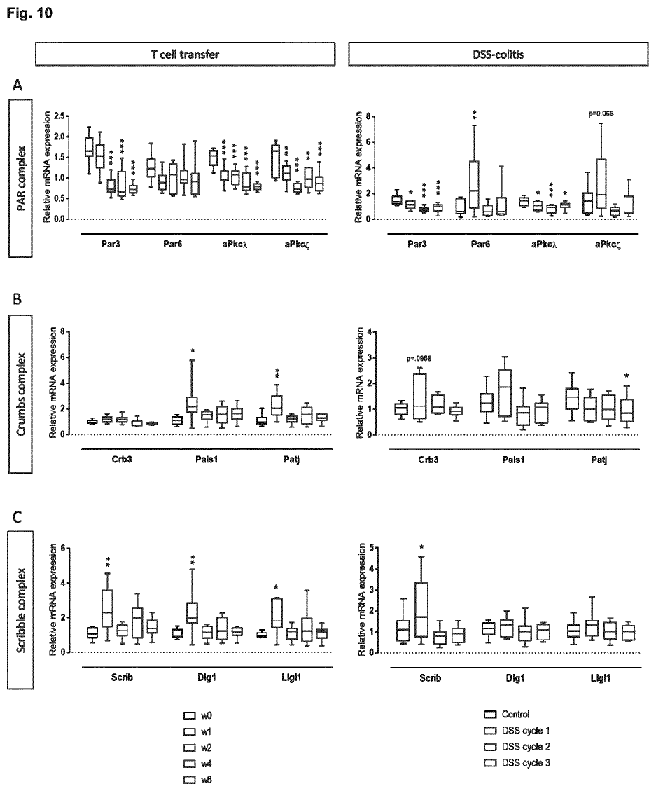

In the T cell transfer model, Muc1 and Muc13 expression were the best factors to discriminate whether mice developed colitis by the adoptive transfer of T cells or were controls (FIG. 11A). In the DSS colitis model, Muc2 expression was found to be the major determinant for identifying mice receiving a DSS treatment, followed by expression of Muc1 and Muc13 (FIG. 11B). Interestingly, increased Muc1 expression correlated significantly with increased intestinal permeability (based on FITC dextran levels in sera) in the T cell transfer model (FIG. 12A), whereas a positive significant correlation between aberrant Muc13 expression and increased intestinal permeability was seen in the DSS model (FIG. 12B). Furthermore, whereas IL-10 was associated with increased permeability and aberrant Muc1 expression in T cell transfer colitis (FIGS. 12A&C), TNF-α positively correlated with intestinal permeability and increased Muc13 expression in DSS-induced colitis (FIGS. 12B&D). Besides, the expression levels of Muc13 also correlated with Muc1 (p=0.013) and Muc2 (p=0.026) expression in the DSS model (data not shown).

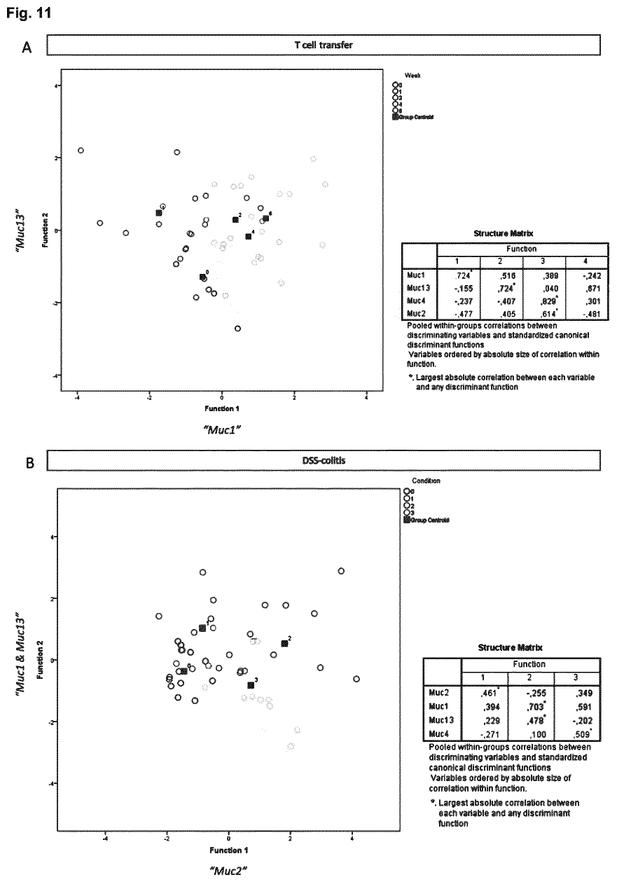

In both colitis models, altered expression of several junctional and polarity proteins correlated significantly with each other (data not shown), further indicating mutual dependence and their involvement in regulating barrier integrity. Moreover, their expression levels could also be used to discriminate between colitis mice and controls (FIG. 13). Furthermore, significant associations between aberrant Muc1, Cldn1, Ocln, Par3 and aPKCζ expression in the T cell transfer model (FIGS. 12E&G) and between aberrant Muc13, Cldn1, Jam2, Tjp2, aPkcζ, Crb3 and Scrib expression in the DSS model (FIGS. 12F&H) further suggested a potential role for Muc1 and Muc13 in intestinal mucosal barrier dysfunction.

### 4. Discussion

The intestinal mucosal barrier plays a critical role in gut health and function. Not only is it a physical barrier between the microbiome, toxins and food antigens in the lumen and the internal host tissues, it also is a dynamic barrier that regulates inflammatory responses. Loss of barrier integrity is generally accepted as a major hallmark in the pathophysiology of IBD. However, whether intestinal barrier dysfunction is a primary contributor to or rather a consequence of intestinal inflammation has not yet been fully elucidated. In this study, we investigated intestinal barrier integrity and inflammation during the course of colitis using the T cell transfer and DSS mouse models. These two models have a different mechanism of initiation of colitis and both are standard IBD models. In both models, increased intestinal permeability in association with an innate inflammatory response, as characterized by increased expression of the pro-inflammatory cytokines TNF-α and IL-1β and decreased expression of the anti-inflammatory cytokine IL-10, was already seen at 1 week post-transfer and after the first DSS administration, and was maintained during the course of disease. Excessive production of TNF-α and IL-1β has been described in IBD patients and these harmful cytokines, produced by T cells, macrophages and neutrophils, are likely to affect intestinal homeostasis leading to further aggravation of inflammation. In our study, increased expression of IL-6 appeared only in later stages of colitis progression. This pro-inflammatory cytokine has been shown to be an important mediator of Th17 cell differentiation, further promoting intestinal inflammation in IBD and modulating intestinal epithelial cells. Also IL-22 was increasingly expressed at the beginning of colitis induction and even at week 6 post-transfer and after the last DSS cycle. This cytokine is normally able to promote mucosal healing in the intestine, but when uncontrolled, it can lead to intestinal inflammation. Based on the above findings, we cannot clearly substantiate whether loss of barrier integrity precedes intestinal inflammation as suggested by several studies, that showed that increased intestinal permeability was present in first-degree relatives of IBD patients before intestinal inflammation occurred. However, expression analysis of junctional proteins and polarity complexes in both our models revealed that most changes already occurred at the beginning of colitis development. This would suggest that loss of barrier integrity is not only a result of an innate inflammatory response but might also be a primary contributor in the pathophysiology of IBD.

The key mediators underlying mucosal barrier dysfunction upon inflammation in IBD still remain to be further elucidated. Often overlooked in intestinal barrier research are the mucins. These heavily glycosylated proteins make up the first part of the barrier, the mucus layer, which is four times thicker than the actual epithelial cell layer and plays an important role in limiting contact between the host and the luminal content. MUC2 is the main component of the secreted mucus layer and provides the first line of defence against invading pathogens and toxins in the intestines. In IBD, this secretory mucin is critical for colonic protection since it has been shown that Muc2^{-/-} mice spontaneously develop colitis. The gradual increase in Muc2 expression seen during the course of colitis in the DSS model can thus be assigned to the host defence to overcome the toxic effects of DSS on the colonic epithelium. Furthermore, this mucin is downregulated in the intestinal mucosae of IBD patients.

Since transmembrane mucins are increasingly expressed in IBD and given their role in signalling pathways involved in cell-cell adhesion and cell differentiation, they are excellent candidates to be involved in the regulation of the barrier function. In our study, expression of the transmembrane Muc1 and Muc1 3 mucins was increased during colitis progression in both models, whereas Muc4 showed variable expression patterns in the inflamed colon. Variable MUC4 expression has also been reported in IBD patients and increased MUC4 expression was mainly observed in UC patients with neoplastic conditions. Altered expression of MUC1 and MUC13 has been shown in the inflamed mucosa of IBD patients and such inappropriate overexpression induced by pro-inflammatory cytokines could lead to aberrant modulation of mucosal epithelial cell inflammatory signalling, which in turn could lead to pathological inflammation. Furthermore, acute DSS studies with knockout animals showed that Muc1^{-/-} mice were resistant to inflammation-induced colitis whereas Muc13^{-/-} mice developed more inflammation compared to wildtype animals. In our DSS model, Muc13 expression was altered in both the acute and chronic phases of DSS-induced colitis. This increase in expression in the more chronic stage of colitis was also confirmed in the T cell transfer model. Unlike MUC1, MUC13 is highly expressed by the intestinal epithelium playing at first a protective role against cytotoxic agents. Furthermore, Sheng and colleagues (Sheng et al., 2012) demonstrated that MUC13 has a pro-inflammatory activity in the intestinal epithelium modulating inflammatory responses induced by TNF-α. Also, in our DSS models, increased TNF-α expression was significantly associated with altered Muc13 expression, further suggesting that expression of this mucin is regulated by TNF-α upon inflammation and thus, the role of this mucin upon chronic colitis should be further investigated. In addition, we were able to correctly annotate individual mice to their experimental group (i.e. control or different time points of colitis) based on Muc1 and Muc13 expression (FIG. 11). Interestingly, three main clusters could be distinguished in both colitis models. In particular, mice that were sacrificed during the initial stages of colitis (after 1 cycle of DSS administration and after 1 week of T cell transfer) were separated from both the control mice and the other experimental groups. Mice that were sacrificed at later time points could clearly be distinguished from control mice yet were more closely associated. These results further indicate the importance of Muc1 and Muc13 during the course of colitis.

To the best of our knowledge, a clear association between increased expression of transmembrane mucins and barrier dysfunction in IBD, has so far never been reported. Here, we found a positive correlation between increased Muc1 and Muc13 expression and increased in vivo intestinal barrier permeability during colitis progression, which was further substantiated by a strong correlation between expression of these mucins and altered expression of barrier mediators, including junctional and polarity proteins. Also observed was a model-specific response for both mucins, which could be explained by the different mechanisms of colitis induction. Whereas colitis in the T cell transfer model is induced by disrupting systemic T cell homeostasis, DSS is toxic to the intestinal epithelium leading to the penetration of luminal bacteria and antigens through the intestinal barrier resulting in a strong innate inflammatory response. Since MUC13 is highly expressed at the healthy intestinal epithelium, its role in modulating the integrity of the intestinal barrier could be related to immediate threats from the external environment. MUC1, on the other hand, is expressed at low levels in the healthy intestine and thus its involvement in barrier dysfunction could be dependent on the infiltration of T lymphocytes upon an inflammatory stimulus. Another possibility is that subtle differences in cytokine secretion could induce specific changes in mucin expression in both models. Although similar cytokine profiles were associated with disease activity in both models, IL-1β was correlated to increased Muc1 expression and in vivo intestinal permeability in the T cell transfer model and TNF-α to increased Muc13 expression and in vivo intestinal permeability in the DSS-induced colitis model. Nevertheless, based on the above findings, we can conclude that aberrantly expressed Muc1 and Muc13 could play a role in modulating intestinal barrier dysfunction during the course of colitis.

Overexpression of transmembrane mucins can result in a repositioning over the whole cell membrane, causing physical hindrance of neighbouring cells to make cell contact⁶. In our control animals, Muc1 and Muc13 were expressed at the apical side of the epithelial membrane, whereas they became generally visible throughout the cell during colitis progression. Transmembrane mucins can affect cell-cell interactions, and thus barrier functionality, in multiple ways. First, via extracellular EGF-like domains and intracellular phosphorylation sites, they can interact with receptor tyrosine kinases, such as ERBB2. Activation of this membrane-bound receptor can then result in a disruption of the PAR polarity complex and subsequent tight junction dysfunction by associating with Par6 and aPKC and blocking the interaction with Par3. In our colitis models, a correlation between increased Muc1 expression and decreased Par3 expression was found suggesting that loss of barrier integrity mediated by Muc1 might be caused by sequestering with ERBB2 and subsequent dissociation of the PAR complex. Interaction of MUC1, but also MUC4 and MUC13, with ERBB2 has been described in many cancer types and the role of ERBB2 in barrier functionality in IBD remains to be further investigated. Second, the cytoplasmic domain of transmembrane mucins can be transported into the nucleus and suppress transcription of crumbs and scribble polarity genes, via interaction with a transcription factor on the promoter of these polarity genes. In this way, loss of cell polarity and tight junction dysfunction can be induced as well. Here, we found a correlation between the expression levels of Muc13, Crb3 and Scrib in the DSS model, highlighting that these mucins could probably also act according to the mechanism described above. Additionally, it has also been described that MUC1 can intracellularly interact with β-catenin, which results in the disruption of the E-cadherin/β-catenin complex and eventually leads to loss of adherens junction stability. In our colitis models, however, increased Muc1 and Muc13 expression was not associated with altered Cdh1 (E-cadherin) expression.

Taken together, the results from our study clearly show the association of aberrant Muc1 and Muc13 expression with intestinal mucosal barrier dysfunction during the course of colitis. A model-specific response was observed, indicating a complex transcriptional regulation of mucin expression that results from the combined effects of the host inflammatory response, the microbiome and the type and course of disease. Nevertheless, the exact mechanisms by which these mucins affect barrier integrity and to prove their functional role in barrier integrity in IBD require further investigation.

Most available therapies in IBD are directed against the inflammatory response. Due to the clinical heterogeneity of these diseases, biologicals are limited in efficacy and safety and still a substantial number of patients fail to respond or obtain full remission. Targeting the barrier, and particularly MUC1 and MUC13, could also have therapeutic potential. These transmembrane mucins have already shown their potential in antibody-based therapy in different cancer types, including colon cancer, making them valuable therapeutic targets in medicine. Furthermore, mucins are highly polymorphic and gene polymorphisms affecting mucin expression have been reported to influence susceptibility towards disease. The presence of genetic differences in mucin genes can result in different mRNA isoforms (i.e. splice variants via alternative splicing) produced from the same mucin gene locus. While most isoforms encode similar biological functions, others have the potential to alter the protein function resulting in progression toward disease¹⁶. So far, only the MUC13-R502S polymorphism has been related to UC and the MUC1-rs3180018 to CD but the MUC1 and MUC13 isoforms associated with IBD remain unknown as well. Inhibiting inflammation-induced MUC1 and MUC13 isoforms to restore intestinal barrier integrity may thus achieve greater efficacy with fewer side effects.

Overall, it is highlighted here that aberrantly expressed Muc1 and Muc13 might be involved in intestinal mucosal barrier dysfunction upon inflammation by affecting tight junction and cell polarity proteins and that they can act as possible targets for novel therapeutic interventions.

### Example 2

### Targeted PacBio Isoform Sequencing to Analyze Isoform Expression of MUC1 and MUC13 in Colonic Biopsies From IBD Patients

### 1. Background

Here, we analyzed the expression of MUC1 and MUC13 isoforms in inflamed and non-inflamed colonic tissue from patients with active IBD to improve our understanding of mucin signaling during chronic inflammation.

2. Methods

### 2.1. IBD Patients and Clinical Specimens

IBD patients that underwent an endoscopy for clinical reasons (i.e. the presence of an acute flare), were recruited via the policlinic of the University Hospital of Antwerp (UZA), Belgium. Colonic biopsies were collected from 3 patients with active disease (1 Crohn's disease, 2 ulcerative colitis) and stored in RNA later at −80° C. until further use. All patients were previously diagnosed with IBD based on bowel complaints, blood and stool tests, radiography, endoscopy and histology. Disease activity was mainly based on the presence of active symptoms and endoscopic and microscopic evaluation of the colon. Prior to endoscopy, informed consent from each patient was obtained. This study was approved by the Ethical Committee of the UZA (Belgian Registration number B300201733423).

### 2.2. RNA Isolation and Quality Control

Total RNA from human colonic tissue stored in RNA later, was extracted using the NucleoSpin® RNA plus kit (Macherey-Nagel) following the manufacturer's instructions. The concentration and purity of the RNA were evaluated using the NanoDrop® ND-1000 UV-Vis Spectrophotometer (Thermo Fisher Scientific) and Qubit Fluorometer (Qubit Broad Range RNA kit, Thermo Fisher Scientific). Quality control of the RNA was performed by capillary electrophoresis using an Agilent 2100 Fragment Analyzer (Agilent).

### 2.3. cDNA Library Preparation and Multiplexing

Initially, 1600-2000 ng of input RNA per sample was used. The reactions from each sample were first labeled with a barcoded oligo dT nucleotide for multiplexing purposes as shown in Table 1. Subsequently, first-strand cDNA synthesis was performed using the SMARTer PCR cDNA synthesis kit (Takara Bio) according to the manufacturer's instructions. The reactions were then diluted 1:10 in Elution Buffer (PacBio) and large-scale amplification was performed using 16 reactions per sample. Each reaction of 50 μL consisted of 10 μL of the diluted cDNA sample, 10 μL 5× PrimeS TAR GXL buffer (Takara Bio), 4 μL dNTP Mix (2.5 mM each), 1 μL 5′ PCR Primer IIA (12 μM), 1 μL PrimeSTAR GXL DNA Polymerase (1.25 U/μL, Takara Bio) and 24 μL nuclease-free water. The samples were then incubated in a thermocyler using the following program: an initial denaturation step at 98° C. for 30 s, followed by 14 cycles of amplification at 98° C. for 10 s, 65° C. for 15 s and 68° C. for 10 min, and a final extension step at 68° C. for 5 min. From these PCR products, two fractions were purified using AMPure magnetic purification beads. After equimolar pooling of both fractions, the samples were finally pooled and the DNA concentration and fragment length evaluated using a Qubit fluorometer (Qubit dsDNA HS kit, ThermoFisher) and an Agilent 2100 Bioanalyzer.

TABLE 1
Barcoded primers used for multiplexing purposes.

| Sample | Barcode | SEQ ID NO | Sequence |
|---|---|---|---|
| P1 colon non-inflamed | dT_BC1001_PB | 61 | AAGCAGTGGTATCAACGCAGAGTACCAC |
| | | | ATATCAGAGTGCGTTTTTTTTTTTTTTT |
| | | | TTTTTTTTTTTTTTTVN |
| P1 colon inflamed | dT_BC1002_PB | 62 | AAGCAGTGGTATCAACGCAGAGTACACA |
| | | | CACAGACTGTGAGTTTTTTTTTTTTTTT |
| | | | TTTTTTTTTTTTTTTVN |
| P2 colon non-inflamed | dT_BC1003_BP | 63 | AAGCAGTGGTATCAACGCAGAGTACACA |
| | | | CATCTCGTGAGAGTTTTTTTTTTTTTTT |
| | | | TTTTTTTTTTTTTTTVN |
| P2 colon inflamed | dT_BC1004_PB | 64 | AAGCAGTGGTATCAACGCAGAGTACCAC |
| | | | GCACACACGCGCGTTTTTTTTTTTTTTT |
| | | | TTTTTTTTTTTTTTTVN |
| P3 colon non-inflamed | dT_BC1005_PB | 65 | AAGCAGTGGTATCAACGCAGAGTACCAC |
| | | | TCGACTCTCGCGTTTTTTTTTTTTTTTT |
| | | | TTTTTTTTTTTTTTTVN |
| P3 colon inflamed | dT_BC1006_PB | 66 | AAGCAGTGGTATCAACGCAGAGTACCAT |
| | | | ATATATCAGCGTGTTTTTTTTTTTTTTT |
| | | | TTTTTTTTTTTTTTTTVN |

In accordance with the IUPAC nucleotide code, N is meant to be any base (A, G, T or C)
and V is meant to be A, C or G.

### 2.4. cDNA Capture Using SeqCap EZ Probes

Initially, 1 μL of SMARTer PCR oligo (1000 μM) and 1 μL PolyT blocker (1000 μM) were added to 1.5 μg cDNA and subsequently dried for 1 hour in a DNA vacuum-concentrator. The cDNA was then hybridized with pre-designed SeqCap EZ probes targeting several mucin coding regions (Table 2 & 3) for 16 hours at 47° C. The captured cDNA was purified using Dynabeads M-270 (Thermo Fisher Scientific) according to the manufacturer's instructions and amplified by preparing a mixture containing 20 μl 10× LA PCR Buffer, 16 μ1 2.5 mM dNTP's, 8.3 SMARTer PCR Oligos (12 μM each), 1.2 μ1 Takara LA Taq DNA polymerase, 50 μl cDNA supplemented with nuclease-free water to an end volume of 200 μl. For the actual PCR, the following program was ran on a thermocycler: an initial denaturation step at 95° C. for 2 min, followed by 11 cycles of amplification at 95° C. for 20s and 68° C. for 10 min, and a final extension step at 72° C. for 10 min. A final clean-up of the amplified captured cDNA was performed using AMPure purification beads. The DNA concentration and fragment length were evaluated using a Qubit fluorometer (Qubit dsDNA HS kit, ThermoFisher) and an Agilent 2100 Bioanalyzer for subsequent SMRTbell library construction.

TABLE 2
Genomic regions targeted with SeqCap EZ probes.

| | | Chromosomal location |
|---|---|---|
| | | (GRCh38/hg38 genome |
| Mucin | Chromosome | annotation) |
| MUC1 | Chr 1 | 155,185,324-155,193,416 |
| MUC2 | Chr11 | 1,074,375-1,111,008 |
| MUC3 - MUC12 - MUC17 | Chr7 | 100,944,420-101,074,859 |
| MUC4 | Chr3 | 195,746,558-195,826,889 |
| MUC5AC - MUC5B | Chr11 | 1,146,953-1,272,672 |
| MUC6 | Chr11 | 1,012,323-1,037,218 |
| MUC13 | Chr3 | 124,905,442-124,940,751 |
| MUC15 | Chr11 | 26,558,532-26,572,763 |
| MUC16 | Chr19 | 8,848,344-9,001,342 |
| MUC20 | Chr3 | 195,720,384-195,738,123 |

TABLE 3
SeqCap EZ probe coverage of targeted mucin regions.

| | Probe coverage | Estimated coverage |
|---|---|---|
| Target Bases Covered | 493.161 (78.7%) | 561.699 (89.7%) |
| Target Bases Not Covered | 133.225 (21.3%) | 64.687 (10.3%) |

### 2.5. SMRTbell Library Construction and Sequencing on the PacBio Sequel System

Using the SMRTbell template prep kit (PacBio), 5 μg of captured cDNA was used for SMRTbell library construction. According to the manufacturer's instructions, the following steps were performed in chronological order: DNA damage repair, end repair, ligation of blunt adapters, Exo III and Exo VII treatment. One intermediate and two final purification steps were performed using AMPure purification beads. The DNA concentration and fragment length were evaluated using a Qubit fluorometer (Qubit dsDNA HS kit, ThermoFisher) and an Agilent 2100 Bioanalyzer for subsequent SMRTbell library construction. Following the instructions on SMRTlink, the Sequel Binding kit (PacBio) and Sequel Sequencing kit (PacBio) were used to dilute the DNA and internal control complexes, anneal the sequencing primer and bind the sequencing polymerase to the SMRTbell templates. Finally, the sample was loaded on a 1M v3 SMRT cell.

### 2.6. Data Analysis

Highly accurate (>99%) polished circular consensus sequencing (ccs) reads were used as initial input for data processing using the command line interface. The lima tool v1.10.0 was used for demultiplexing and primer removal. Subsequently, the isoseq3 v3.2.2 package was used for further read processing to generate high quality mRNA transcripts. First, the refine tool was used for trimming of Poly(A) tails and identification and removal of concatemers. The data of the individual samples were then pooled together according to the condition (i.e. 3 samples from non-inflamed tissue, 3 samples from inflamed tissue or all samples together) and analyzed in parallel. The isoseq3 cluster algorithm was used for transcript clustering. Minimap2 was used for the alignment of the processed reads to the human reference genome (GRCh38). After mapping, ToFU scripts from the cDNA_Cupcake GitHub repository were used to collapse redundant isoforms (minimal alignment coverage and minimal alignment identity set at 0.95), identify associated count information and filter away 5′ degraded isoforms. Finally, the SQANTI2 tool was used for extensive characterization of MUC1 and MUC13 mRNA isoforms. The eventual isoforms were then further inspected by visualization in the Integrative Genomics Viewer (IGV) version 2.8.0 and by the analysis of the classification and junction files in Excel.

### 3. Results

### 3.1. Patient and Sample Characteristics

The samples were collected from the colon of 3 patients with known and active IBD, of which two were diagnosed with ulcerative colitis and one with Crohn's disease. Year of diagnosis and medication use was different for all patients. During endoscopy, the samples were collected from a macroscopically inflamed region in the colon and from an adjacent macroscopically non-inflamed region. A detailed overview of the patient characteristics as well as the location of the colon biopsies is shown in table 4.

TABLE 4
Summary of patient characteristics and primary disease
location from which biopsies were collected.

| | | | | Years | Primary | |
|---|---|---|---|---|---|---|
| | | | | since | medication | Primary disease |
| Patient | Sex | Age | Diagnosis | diagnosis | use | location |
| Patient 1 | Female | 34 | Crohn's disease | 20 | Remicade | Rectum |
| Patient 2 | Female | 36 | Ulcerative colitis | 10 | No | Rectum/anus |
| Patient 3 | Female | 45 | Ulcerative colitis | 3 | Mesalamine | Sigmoid and |
| | | | | | | descending colon |

### 3.2. General Features of Sequencing Run

Sequencing of all samples initially generated 103 699 ccs reads. Sequencing yield and read quality was high and comparable across all samples. The average read length was 2082 bp. 24592 (24%) reads were lost during primer removal and demultiplexing as a consequence of undesired barcoded primer combinations. After clustering, 55312 reads were remained corresponding to 6617 different transcripts. As visual analysis of targeted mucin regions in IGV showed complete and dense coverage of the full genomic region of only MUC1 and MUC13, further analysis was limited to these two mucin glycoproteins.

### 3.3. MUC1 Isoforms

Targeted PacBio isoform sequencing revealed the identification of both known and novel MUC1 isoforms in colonic tissue from IBD patients that were all found to be coding transcripts (FIG. 14 & Table 5). In particular, 7 alternative mRNA transcripts (=isoforms) were found in both non-inflamed and inflamed colonic tissue, of which 1 (PB.136.39) matched to a known isoform (ENST00000462317.5) and 6 had not been described elsewhere. Interestingly, from these alternative transcripts, 3 were increased in expression based on the read counts in the inflamed tissue as compared to the non-inflamed tissue (PB.136.1, PB.136.25, PB.136.28). Additionally, 2 other novel isoforms were found which were only reported in non-inflamed colonic tissue, whereas in the inflamed colonic tissue, 1 known (PB.136.19; ENST00000368390.7) and 11 novel alternative transcripts were found. Interestingly, 2 newly identified isoforms showed dominant expression in the inflamed tissue (PB.136.2, PB.136.15). Concerning the overall exonic structure of the alternative transcripts, no transcripts were found that contained exon 3 to 5 (VNTR). Exon 2 (VNTR) and exon 6 (SEA domain) were most prone to alternative splicing in both non-inflamed and inflamed colonic tissue (FIG. 14 & Table 5). All novel alternative transcripts found resulted from the partial retention of intronic regions (Table 5). A detailed overview of splice junctions can be found in supplementary table S2.

The results of these limited number of samples clearly shows that different alternative transcripts of MUC1 are formed in the colon and that inflammation stimulates alternative splicing as well as increasing the expression of particular transcripts. This is the first study that highlights the potential importance of MUC1 isoforms in IBD. Only in cancer research, a few papers investigating the pathogenic significance of MUC1 splice variants are available. More specifically, it has been shown that different MUC1 isoforms might interact together to form a ligand-receptor complex, associate with other host receptors or influence cytokine expression mediating inflammatory signaling pathways (Zaretsky et al., 2006). Alternative splicing of MUCI isoforms was also shown to be cancer-type dependent and able to distinguish cancer samples from benign samples (Obermair et al., 2002). In breast cancer, for instance, it has been described that a shorter MUC1 isoform was specifically expressed in tumor tissue but not in the adjacent healthy tissue (Zrihan-Licht et al., 1994), whereas estrogen treatment induced the expression of another variant (Zartesky et al., 2006). All this highlight the intriguing complexity and biological role of alternative splicing.

TABLE 5
Detailed overview of characteristics of MUC1 mRNA
isoforms in colonic biopsies from IBD patients
BOTH CONDITIONS

| | | | | | | Main mechanism | |
|---|---|---|---|---|---|---|---|
| | | Length | | | | of alternative | |
| Isoform ID | Chrom | (bp) | Exons | Coding | Transcript | splicing | Counts |
| PB.136.1 | chr1 | 1712 | 8 | Coding | Novel | Intron retention | NI: 3 |
| | | | | | | | I: 11 |
| PB.136.23 | chr1 | 1257 | 7 | Coding | Novel | Intron retention | NI: 8 |
| | | | | | | | I: 9 |
| PB.136.26 | chr1 | 1619 | 8 | Coding | Novel | Intron retention | NI: 2 |
| | | | | | | | I: 5 |
| PB.136.28 | chr1 | 1551 | 8 | Coding | Novel | Intron retention | NI: 8 |
| | | | | | | | I: 21 |
| PB.136.25 | chr1 | 2306 | 6 | Coding | Novel | Intron retention | NI: 5 |
| | | | | | | | I: 15 |
| PB.136.39 | chr1 | 1377 | 8 | Coding | ENST00000462317.5 | Multi-exon | NI: 3 |
| | | | | | | | I: 3 |
| PB.136.5 | chr1 | 1090 | 6 | Coding | Novel | Intron retention | NI: 2 |
| | | | | | | | I: 3 |

| | | | | | | | |
|---|---|---|---|---|---|---|---|
| | | Length | | | | | |
| Isoform ID | Chrom | (bp) | Exons | Coding | Transcript | subcategory | Counts |

NON-INFLAMED

| | | | | | | | |
|---|---|---|---|---|---|---|---|
| PB.136.9 | chr1 | 1497 | 8 | Coding | Novel | Intron retention | 7 |
| PB.136.22 | chr1 | 1493 | 8 | Coding | Novel | Intron retention | 2 |

INFLAMED

| | | | | | | | |
|---|---|---|---|---|---|---|---|
| PB.136.2 | chr1 | 1652 | 8 | Coding | Novel | Intron retention | 30 |
| PB.136.4 | chr1 | 1470 | 8 | Coding | Novel | Intron retention | 2 |
| PB.136.18 | chr1 | 1233 | 8 | Coding | Novel | Intron retention | 2 |
| PB.136.15 | chr1 | 1526 | 8 | Coding | Novel | Intron retention | 24 |
| PB.136.14 | chr1 | 1564 | 8 | Coding | Novel | Intron retention | 3 |
| PB.136.19 | chr1 | 1141 | 8 | Coding | ENST00000368390.7 | Multi-exon | 3 |
| PB.136.21 | chr1 | 1590 | 8 | Coding | Novel | Intron retention | 3 |
| PB.136.29 | chr1 | 1493 | 8 | Coding | Novel | Intron retention | 2 |
| PB.136.37 | chr1 | 1640 | 8 | Coding | Novel | Intron retention | 2 |
| PB.136.38 | chr1 | 1583 | 8 | Coding | Novel | Intron retention | 5 |
| PB.136.6 | chr1 | 1055 | 6 | Coding | Novel | Intron retention | 3 |
| PB.136.24 | chr1 | 1088 | 7 | Coding | Novel | Intron retention | 2 |

### 3.4. MUC13 Isoforms

Twenty-one alternative MUC13 mRNA transcripts were found in colonic tissue from IBD patients (FIG. 15 & Table 6). Of these, 17 transcripts were identified as being coding isoforms and 4 as non-coding splice variants. Such long untranslated mucin isoforms can function similar to long noncoding RNA and act as a scaffold for assembly of multimeric protein complexes involved in the regulation of cellular processes. Importantly, the full-length known isoform (ENST00000616727.4) was present in both conditions but was highly upregulated in the inflamed colonic tissue (Table 6). In both conditions, 3 additional isoforms were found that had not been reported previously. Other isoforms showed a condition-specific expression pattern. More specifically, 4 mRNA isoforms were uniquely found in the non-inflamed tissue, whereas 13 mRNA isoforms were only reported in the inflamed colonic tissue. Several mechanisms of alternative splicing were identified concerning MUC13 isoforms. Exon skipping was observed in two alternative transcripts in the inflamed colon (i.e. exon 9 (EGF-like) and 10 (TMD) in PB.1087.32; exon 9 (EGF-like), 10 (TMD) and 11 (CT) in PB.1087.20). Some mono-exonic transcripts were found that resulted from intron retention in the genomic region coding for the ECD (i.e. PB.1087.50, PB.1087.53, PB.1087.58, PB.1087.61). The other isoforms resulted from more subtle recombinations using both known and novel splice sites mainly in the ECD-coding regions of MUC13 (FIG. 15 & Table 6). A detailed overview of all splice junctions can be found in Supplementary table S3.

To our knowledge, the heterogeneity of MUC13 isoform expression during inflammation and cancer has not been studied in much detail before. Here, evidence is provided that MUC13 is alternatively spliced in both non-inflamed and inflamed colonic tissue from IBD patients.

TABLE 6
Detailed overview of characteristics of MUC13 mRNA isoforms in colonic biopsies from IBD patients

| | | | | | | | | |
|---|---|---|---|---|---|---|---|---|
| | | | | | | | Main | |
| | | | | | | | mechanism of | |
| | | | Length | | | | alternative | |
| Isoform ID | Chrom | Strand | (bp) | Exons | Coding | Transcript | splicing | Counts |

BOTH CONDITIONS

| | | | | | | | | |
|---|---|---|---|---|---|---|---|---|
| PB.1087.17 | chr3 | — | 2878 | 12 | Coding | ENST00000616727.4 | Constitutive | NI: 518 |
| | | | | | | | | I: 936 |
| PB.1087.22 | chr3 | — | 2830 | 13 | Coding | Novel | At least one | NI: 3 |
| | | | | | | | novel splicesite | I: 7 |
| PB.1087.30 | chr3 | — | 2859 | 12 | Coding | Novel | At least one | NI: 2 |
| | | | | | | | novel splicesite | I: 2 |
| PB.1087.55 | chr3 | — | 5414 | 3 | Coding | Novel | Intron retention | NI: 2 |
| | | | | | | | | I: 4 |

NON-INFLAMED

| | | | | | | | | |
|---|---|---|---|---|---|---|---|---|
| PB.1087.18 | chr3 | — | 2725 | 13 | Coding | Novel | At least one | 2 |
| | | | | | | | novel splicesite | |
| PB.1087.50 | chr3 | — | 5304 | 1 | Non-coding | Novel | Mono-exon/ | 2 |
| | | | | | | | intron retention | |
| PB.1087.61 | chr3 | — | 5106 | 1 | Coding | Novel | Mono-exon/ | 2 |
| | | | | | | | intron retention | |
| PB.1087.64 | chr3 | — | 3860 | 2 | Coding | Novel | At least one | 3 |
| | | | | | | | novel splicesite | |

INFLAMED

| | | | | | | | | |
|---|---|---|---|---|---|---|---|---|
| PB.1087.6 | chr3 | — | 2243 | 10 | Coding | Novel | At least one | 2 |
| | | | | | | | novel splicesite | |
| PB.1087.63 | chr3 | — | 3962 | 2 | Coding | Novel | At least one | 2 |
| | | | | | | | novel splicesite | |
| PB.1087.21 | chr3 | — | 3195 | 13 | Coding | Novel | At least one | 2 |
| | | | | | | | novel splicesite | |
| PB.1087.20 | chr3 | — | 1979 | 9 | Coding | Novel | At least one | 2 |
| | | | | | | | novel splicesite | |
| PB.1087.25 | chr3 | — | 2671 | 11 | Coding | Novel | At least one | 2 |
| | | | | | | | novel splicesite | |
| PB.1087.68 | chr3 | — | 2643 | 2 | Coding | Novel | At least one | 2 |
| | | | | | | | novel splicesite | |
| PB.1087.32 | chr3 | — | 2754 | 10 | Coding | Novel | Novel combination of | 8 |
| | | | | | | | known splicesites | |
| PB.1087.27 | chr3 | — | 2328 | 12 | Coding | Novel | At least one | 2 |
| | | | | | | | novel splicesite | |
| PB.1087.31 | chr3 | — | 2795 | 13 | Coding | Novel | At least one | 2 |
| | | | | | | | novel splicesite | |
| PB.1087.52 | chr3 | — | 3622 | 4 | Coding | Novel | Intron retention | 2 |
| PB.1087.53 | chr3 | — | 2303 | 1 | Non-coding | Novel | Mono-exon/ | 2 |
| | | | | | | | intron retention | |
| PB.1087.58 | chr3 | — | 2362 | 1 | Non-coding | Novel | Mono-exon/ | 4 |
| | | | | | | | intron retention | |
| PB.1087.56 | chr3 | — | 5246 | 2 | Coding | Novel | Intron retention | 3 |

### 4. Concluding Remarks

Based on the PacBio isoform sequencing data gathered from a limited number of samples, we were able to identify both known and novel mRNA isoforms of MUC1 and MUC13 in non-inflamed and inflamed colonic tissue from IBD patients. Alternative splicing of MUC1 and MUC13 mucin genes was clearly increased upon inflammation. Although some isoforms were found in both inflamed and non-inflamed tissue, several other isoforms were uniquely attributed to inflammation.

In conclusion, mucin isoform expression is altered upon inflammation in IBD patients, highlighting its potential for disease surveillance or treatment. Moreover, these novel insights could be extrapolated to other inflammatory diseases and cancer that involve a dysfunctional mucosal epithelial barrier. The unexplored world of mucin isoforms provides thus a unique opportunity to understand their biological significance, utility as biomarker and pathology-specific targeting.

BOTH CONDITIONS

| | | | junction_ | genomic_ | genomic_ | junction_ | splice_ | |
|---|---|---|---|---|---|---|---|---|
| isoform | Chrom | strand | number | start_coord | end_coord | category | site | canonical |
| PB.136.1 | chr1 | — | junction_1 | 155186210 | 155187224 | known | GTAG | canonical |
| PB.136.1 | chr1 | — | junction_2 | 155187375 | 155187454 | known | GTAG | canonical |
| PB.136.1 | chr1 | — | junction_3 | 155187577 | 155187721 | known | GTAG | canonical |
| PB.136.1 | chr1 | — | junction_4 | 155187859 | 155188007 | known | GTAG | canonical |
| PB.136.1 | chr1 | — | junction_5 | 155188064 | 155188162 | known | GTAG | canonical |
| PB.136.1 | chr1 | — | junction_6 | 155188528 | 155191938 | novel | CCAG | non_canonical |
| PB.136.1 | chr1 | — | junction_7 | 155192311 | 155192785 | known | GTAG | canonical |
| PB.136.23 | chr1 | — | junction_1 | 155186210 | 155187224 | known | GTAG | canonical |
| PB.136.23 | chr1 | — | junction_2 | 155187375 | 155187454 | known | GTAG | canonical |
| PB.136.23 | chr1 | — | junction_3 | 155187577 | 155187721 | known | GTAG | canonical |
| PB.136.23 | chr1 | — | junction_4 | 155187859 | 155188007 | known | GTAG | canonical |
| PB.136.23 | chr1 | — | junction_5 | 155188064 | 155188162 | known | GTAG | canonical |
| PB.136.23 | chr1 | — | junction_6 | 155188452 | 155192787 | novel | AGAG | non_canonical |
| PB.136.26 | chr1 | — | junction_2 | 155187375 | 155187454 | known | GTAG | canonical |
| PB.136.26 | chr1 | — | junction_3 | 155187577 | 155187721 | known | GTAG | canonical |
| PB.136.26 | chr1 | — | junction_4 | 155187859 | 155188007 | known | GTAG | canonical |
| PB.136.26 | chr1 | — | junction_5 | 155188064 | 155188162 | known | GTAG | canonical |
| PB.136.26 | chr1 | — | junction_6 | 155188538 | 155192008 | novel | ACCC | non_canonical |
| PB.136.26 | chr1 | — | junction_7 | 155192284 | 155192785 | known | GTAG | canonical |
| PB.136.28 | chr1 | — | junction_1 | 155186210 | 155187224 | known | GTAG | canonical |
| PB.136.28 | chr1 | — | junction_2 | 155187375 | 155187454 | known | GTAG | canonical |
| PB.136.28 | chr1 | — | junction_3 | 155187577 | 155187721 | known | GTAG | canonical |
| PB.136.28 | chr1 | — | junction_4 | 155187859 | 155188007 | known | GTAG | canonical |
| PB.136.28 | chr1 | — | junction_5 | 155188064 | 155188162 | known | GTAG | canonical |
| PB.136.28 | chr1 | — | junction_6 | 155188452 | 155192017 | novel | GGAG | non_canonical |
| PB.136.28 | chr1 | — | junction_7 | 155192311 | 155192785 | known | GTAG | canonical |
| PB.136.25 | chr1 | — | junction_1 | 155187375 | 155187454 | known | GTAG | canonical |
| PB.136.25 | chr1 | — | junction_2 | 155187577 | 155187721 | known | GTAG | canonical |
| PB.136.25 | chr1 | — | junction_3 | 155187859 | 155188007 | known | GTAG | canonical |
| PB.136.25 | chr1 | — | junction_4 | 155188064 | 155188162 | known | GTAG | canonical |
| PB.136.25 | chr1 | — | junction_5 | 155188557 | 155191967 | novel | GCAG | canonical |
| PB.136.39 | chr1 | — | junction_1 | 155186210 | 155186729 | known | GTAG | canonical |
| PB.136.39 | chr1 | — | junction_2 | 155186805 | 155187224 | known | GTAG | canonical |
| PB.136.39 | chr1 | — | junction_3 | 155187375 | 155187454 | known | GTAG | canonical |
| PB.136.39 | chr1 | — | junction_4 | 155187577 | 155187721 | known | GTAG | canonical |
| PB.136.39 | chr1 | — | junction_5 | 155187859 | 155188007 | known | GTAG | canonical |
| PB.136.39 | chr1 | — | junction_6 | 155188064 | 155188162 | known | GTAG | canonical |
| PB.136.39 | chr1 | — | junction_7 | 155188541 | 155192863 | novel | CCCC | non_canonical |
| PB.136.5 | chr1 | — | junction_1 | 155186210 | 155187224 | known | GTAG | canonical |
| PB.136.5 | chr1 | — | junction_2 | 155187375 | 155187454 | known | GTAG | canonical |
| PB.136.5 | chr1 | — | junction_3 | 155187545 | 155187721 | known | GTAG | canonical |
| PB.136.5 | chr1 | — | junction_4 | 155187859 | 155188007 | known | GTAG | canonical |
| PB.136.5 | chr1 | — | junction_78785 | 155188064 | 155188162 | known | GTAG | canonical |

NON INFLAMED

| | Chromo- | | junction_ | genomic_ | genomic | junction_ | splice_ | |
|---|---|---|---|---|---|---|---|---|
| isoform | some | strand | number | start_coord | end_coord | categoty | site | canonical |
| PB.136.9 | chr1 | — | junction_1 | 155186210 | 155187224 | known | GTAG | canonical |
| PB.136.9 | chr1 | — | junction_2 | 155187375 | 155187454 | known | GTAG | canonical |
| PB.136.9 | chr1 | — | junction_3 | 155187577 | 155187721 | known | GTAG | canonical |
| PB.136.9 | chr1 | — | junction_4 | 155187859 | 155188007 | known | GTAG | canonical |
| PB.136.9 | chr1 | — | junction_5 | 155188064 | 155188162 | known | GTAG | canonical |
| PB.136.9 | chr1 | — | junction_6 | 155188533 | 155192128 | novel | ACCC | non_canonical |
| PB.136.9 | chr1 | — | junction_7 | 155192284 | 155192785 | known | GTAG | canonical |
| PB.136.22 | chr1 | — | junction_1 | 155186210 | 155187224 | known | GTAG | canonical |
| PB.136.22 | chr1 | — | junction_2 | 155187375 | 155187454 | known | GTAG | canonical |
| PB.136.22 | chr1 | — | junction_3 | 155187577 | 155187721 | known | GTAG | canonical |
| PB.136.22 | chr1 | — | junction_4 | 155187859 | 155188007 | known | GTAG | canonical |
| PB.136.22 | chr1 | — | junction_5 | 155188064 | 155188162 | known | GTAG | canonical |
| PB.136.22 | chr1 | — | junction_6 | 155188467 | 155192028 | novel | GGAA | non_canonical |
| PB.136.22 | chr1 | — | junction_7 | 155192248 | 155192785 | novel | GTAG | canonical |

INFLAMED

| | chromo- | | junction_ | genomic_ | genomic | junction_ | splice_ | |
|---|---|---|---|---|---|---|---|---|
| isoform | some | strand | number | start_coord | end_coord | category | site | canonical |
| PB.136.2 | chr1 | — | junction_1 | 155186210 | 155187224 | known | GTAG | canonical |
| PB.136.2 | chr1 | — | junction_2 | 155187375 | 155187454 | known | GTAG | canonical |
| PB.136.2 | chr1 | — | junction_3 | 155187577 | 155187721 | known | GTAG | canonical |
| PB.136.2 | chr1 | — | junction_4 | 155187859 | 155188007 | known | GTAG | canonical |
| PB.136.2 | chr1 | — | junction_5 | 155188064 | 155188162 | known | GTAG | canonical |
| PB.136.2 | chr1 | — | junction_6 | 155188538 | 155192008 | novel | ACCC | non_canonical |
| PB.136.2 | chr1 | — | junction_7 | 155192311 | 155192785 | known | GTAG | canonical |
| PB.136.4 | chr1 | — | junction_1 | 155186210 | 155187224 | known | GTAG | canonical |
| PB.136.4 | chr1 | — | junction_2 | 155187375 | 155187454 | known | GTAG | canonical |
| PB.136.4 | chr1 | — | junction_3 | 155187577 | 155187721 | known | GTAG | canonical |
| PB.136.4 | chr1 | — | junction_4 | 155187859 | 155188007 | known | GTAG | canonical |
| PB.136.4 | chr1 | — | junction_5 | 155188064 | 155188162 | known | GTAG | canonical |
| PB.136.4 | chr1 | — | junction_6 | 155188528 | 155192153 | novel | CCAG | non_canonical |
| PB.136.4 | chr1 | — | junction_7 | 155192284 | 155192785 | known | GTAG | canonical |
| PB.136.18 | chr1 | — | junction_1 | 155186210 | 155187224 | known | GTAG | canonical |
| PB.136.18 | chr1 | — | junction_2 | 155187375 | 155187454 | known | GTAG | canonical |
| PB.136.18 | chr1 | — | junction_3 | 155187577 | 155187721 | known | GTAG | canonical |
| PB.136.18 | chr1 | — | junction_4 | 155187859 | 155188007 | known | GTAG | canonical |
| PB.136.18 | chr1 | — | junction_5 | 155188064 | 155188162 | known | GTAG | canonical |
| PB.136.18 | chr1 | — | junction_6 | 155188375 | 155192244 | novel | GATG | non_canonical |
| PB.136.18 | chr1 | — | junction_7 | 155192284 | 155192785 | known | GTAG | canonical |
| PB.136.15 | chr1 | — | junction_1 | 155186210 | 155187224 | known | GTAG | canonical |
| PB.136.15 | chr1 | — | junction_2 | 155187375 | 155187454 | known | GTAG | canonical |
| PB.136.15 | chr1 | — | junction_3 | 155187577 | 155187721 | known | GTAG | canonical |
| PB.136.15 | chr1 | — | junction_4 | 155187859 | 155188007 | known | GTAG | canonical |
| PB.136.15 | chr1 | — | junction_5 | 155188064 | 155188162 | known | GTAG | canonical |
| PB.136.15 | chr1 | — | junction_6 | 155188452 | 155192017 | novel | GGAG | non_canonical |
| PB.136.15 | chr1 | — | junction_7 | 155192284 | 155192785 | known | GTAG | canonical |
| PB.136.14 | chr1 | — | junction_1 | 155186210 | 155187224 | known | GTAG | canonical |
| PB.136.14 | chr1 | — | junction_2 | 155187375 | 155187454 | known | GTAG | canonical |
| PB.136.14 | chr1 | — | junction_3 | 155187577 | 155187721 | known | GTAG | canonical |
| PB.136.14 | chr1 | — | junction_4 | 155187859 | 155188007 | known | GTAG | canonical |
| PB.136.14 | chr1 | — | junction_5 | 155188064 | 155188162 | known | GTAG | canonical |
| PB.136.14 | chr1 | — | junction_6 | 155188471 | 155192025 | novel | CAGC | non_canonical |
| PB.136.14 | chr1 | — | junction_7 | 155192311 | 155192785 | known | GTAG | canonical |
| PB.136.19 | chr1 | — | junction_1 | 155186210 | 155187224 | known | GTAG | canonical |
| PB.136.19 | chr1 | — | junction_2 | 155187375 | 155187454 | known | GTAG | canonical |
| PB.136.19 | chr1 | — | junction_3 | 155187577 | 155187721 | known | GTAG | canonical |
| PB.136.19 | chr1 | — | junction_4 | 155187859 | 155188007 | known | GTAG | canonical |
| PB.136.19 | chr1 | — | junction_5 | 155188064 | 155188162 | known | GTAG | canonical |
| PB.136.19 | chr1 | — | junction_6 | 155188232 | 155192182 | known | GTAG | canonical |
| PB.136.19 | chr1 | — | junction_7 | 155192284 | 155192785 | known | GTAG | canonical |
| PB.136.21 | chr1 | — | junction_1 | 155186210 | 155187224 | known | GTAG | canonical |
| PB.136.21 | chr1 | — | junction_2 | 155187375 | 155187454 | known | GTAG | canonical |
| PB.136.21 | chr1 | — | junction_3 | 155187577 | 155187721 | known | GTAG | canonical |
| PB.136.21 | chr1 | — | junction_4 | 155187859 | 155188007 | known | GTAG | canonical |
| PB.136.21 | chr1 | — | junction_5 | 155188064 | 155188162 | known | GTAG | canonical |
| PB.136.21 | chr1 | — | junction_6 | 155188467 | 155191967 | novel | GCAA | non_canonical |
| PB.136.21 | chr1 | — | junction_7 | 155192284 | 155192785 | known | GTAG | canonical |
| PB.136.29 | chr1 | — | junction_1 | 155186210 | 155187224 | known | GTAG | canonical |
| PB.136.29 | chr1 | — | junction_2 | 155187375 | 155187454 | known | GTAG | canonical |
| PB.136.29 | chr1 | — | junction_3 | 155187577 | 155187721 | known | GTAG | canonical |
| PB.136.29 | chr1 | — | junction_4 | 155187859 | 155188007 | known | GTAG | canonical |
| PB.136.29 | chr1 | — | junction_5 | 155188064 | 155188162 | known | GTAG | canonical |
| PB.136.29 | chr1 | — | junction_6 | 155188528 | 155192153 | novel | CCAG | non_canonical |
| PB.136.29 | chr1 | — | junction_7 | 155192311 | 155192785 | known | GTAG | canonical |
| PB.136.37 | chr1 | — | junction_1 | 155186210 | 155187224 | known | GTAG | canonical |
| PB.136.37 | chr1 | — | junction_2 | 155187375 | 155187454 | known | GTAG | canonical |
| PB.136.37 | chr1 | — | junction_3 | 155187577 | 155187721 | known | GTAG | canonical |
| PB.136.37 | chr1 | — | junction_4 | 155187859 | 155188007 | known | GTAG | canonical |
| PB.136.37 | chr1 | — | junction_5 | 155188064 | 155188162 | known | GTAG | canonical |
| PB.136.37 | chr1 | — | junction_6 | 155188580 | 155191990 | novel | GTGT | non_canonical |
| PB.136.37 | chr1 | — | junction_7 | 155192248 | 155192785 | novel | GTAG | canonical |
| PB.136.38 | chr1 | — | junction_1 | 155186210 | 155187224 | known | GTAG | canonical |
| PB.136.38 | chr1 | — | junction_2 | 155187375 | 155187454 | known | GTAG | canonical |
| PB.136.38 | chr1 | — | junction_3 | 155187577 | 155187721 | known | GTAG | canonical |
| PB.136.38 | chr1 | — | junction_4 | 155187859 | 155188007 | known | GTAG | canonical |
| PB.136.38 | chr1 | — | junction_5 | 155188064 | 155188162 | known | GTAG | canonical |
| PB.136.38 | chr1 | — | junction_6 | 155188580 | 155192110 | novel | GTGT | non_canonical |
| PB.136.38 | chr1 | — | junction_7 | 155192311 | 155192785 | known | GTAG | canonical |
| PB.136.6 | chr1 | — | junction_1 | 155186210 | 155187224 | known | GTAG | canonical |
| PB.136.6 | chr1 | — | junction_2 | 155187375 | 155187454 | known | GTAG | canonical |
| PB.136.6 | chr1 | — | junction_3 | 155187577 | 155187721 | known | GTAG | canonical |
| PB.136.6 | chr1 | — | junction_4 | 155187804 | 155188007 | novel | GTAT | non_canonical |
| PB.136.6 | chr1 | — | junction_5 | 155188064 | 155188162 | known | GTAG | canonical |
| PB.136.24 | chr1 | — | junction_1 | 155185989 | 155186052 | novel | CTCC | non_canonical |
| PB.136.24 | chr1 | — | junction_2 | 155186210 | 155187224 | known | GTAG | canonical |
| PB.136.24 | chr1 | — | junction_3 | 155187375 | 155187454 | known | GTAG | canonical |
| PB.136.24 | chr1 | — | junction_4 | 155187577 | 155187721 | known | GTAG | canonical |
| PB.136.24 | chr1 | — | junction_5 | 155187859 | 155188007 | known | GTAG | canonical |
| PB.136.24 | chr1 | — | junction_6 | 155188064 | 155188162 | known | GTAG | canonical |

BOTH CONDITIONS

| | chromo- | | junction_ | genomic_ | genomic | junction_ | splice_ | |
|---|---|---|---|---|---|---|---|---|
| Isoform | some | strand | number | start_coord | end_coord | category | site | Canonical |
| PB.1087.17 | chr3 | — | junction_1 | 124906743 | 124908146 | known | GTAG | canonical |
| PB.1087.17 | chr3 | — | junction_2 | 124908349 | 124910414 | known | GTAG | canonical |
| PB.1087.17 | chr3 | — | junction_3 | 124910500 | 124912103 | known | GTAG | canonical |
| PB.1087.17 | chr3 | — | junction_4 | 124912142 | 124913110 | known | GTAG | canonical |
| PB.1087.17 | chr3 | — | junction_5 | 124913241 | 124913561 | known | GTAG | canonical |
| PB.1087.17 | chr3 | — | junction_6 | 124913682 | 124916316 | known | GTAG | canonical |
| PB.1087.17 | chr3 | — | junction_7 | 124916481 | 124920233 | known | GTAG | canonical |
| PB.1087.17 | chr3 | — | junction_8 | 124920290 | 124922196 | known | GTAG | canonical |
| PB.1087.17 | chr3 | — | junction_9 | 124922304 | 124923526 | known | GTAG | canonical |
| PB.1087.17 | chr3 | — | junction_10 | 124923650 | 124927531 | known | GTAG | canonical |
| PB.1087.17 | chr3 | — | junction_11 | 124927994 | 124934660 | known | GTAG | canonical |
| PB.1087.22 | chr3 | — | junction_1 | 124906743 | 124908146 | known | GTAG | canonical |
| PB.1087.22 | chr3 | — | junction_2 | 124908349 | 124910414 | known | GTAG | canonical |
| PB.1087.22 | chr3 | — | junction_3 | 124910500 | 124912103 | known | GTAG | canonical |
| PB.1087.22 | chr3 | — | junction_4 | 124912142 | 124913110 | known | GTAG | canonical |
| PB.1087.22 | chr3 | — | junction_5 | 124913241 | 124913561 | known | GTAG | canonical |
| PB.1087.22 | chr3 | — | junction_6 | 124913682 | 124916316 | known | GTAG | canonical |
| PB.1087.22 | chr3 | — | junction_7 | 124916481 | 124920233 | known | GTAG | canonical |
| PB.1087.22 | chr3 | — | junction_8 | 124920290 | 124922196 | known | GTAG | canonical |
| PB.1087.22 | chr3 | — | junction_9 | 124922304 | 124923526 | known | GTAG | canonical |
| PB.1087.22 | chr3 | — | junction_10 | 124923650 | 124927531 | known | GTAG | canonical |
| PB.1087.22 | chr3 | — | junction_11 | 124927748 | 124927795 | novel | CATA | non_canonical |
| PB.1087.22 | chr3 | — | junction_12 | 124927994 | 124934660 | known | GTAG | canonical |
| PB.1087.30 | chr3 | — | junction_1 | 124906743 | 124908146 | known | GTAG | canonical |
| PB.1087.30 | chr3 | — | junction_2 | 124908349 | 124910414 | known | GTAG | canonical |
| PB.1087.30 | chr3 | — | junction_3 | 124910500 | 124912103 | known | GTAG | canonical |
| PB.1087.30 | chr3 | — | junction_4 | 124912142 | 124913110 | known | GTAG | canonical |
| PB.1087.30 | chr3 | — | junction_5 | 124913241 | 124913561 | known | GTAG | canonical |
| PB.1087.30 | chr3 | — | junction_6 | 124913682 | 124916316 | known | GTAG | canonical |
| PB.1087.30 | chr3 | — | junction_7 | 124916463 | 124920233 | novel | GTAG | canonical |
| PB.1087.30 | chr3 | — | junction_8 | 124920290 | 124922196 | known | GTAG | canonical |
| PB.1087.30 | chr3 | — | junction_9 | 124922304 | 124923526 | known | GTAG | canonical |
| PB.1087.30 | chr3 | — | junction_10 | 124923650 | 124927531 | known | GTAG | canonical |
| PB.1087.30 | chr3 | — | junction_11 | 124927994 | 124934660 | known | GTAG | canonical |
| PB.1087.55 | chr3 | — | junction_1 | 124922615 | 124922693 | novel | GTTC | non_canonical |
| PB.1087.55 | chr3 | — | junction_2 | 124927994 | 124934660 | known | GTAG | canonical |

NON INFLAMED

| | chromo- | | junction_ | genomic_ | genomic_ | junction_ | splice_ | |
|---|---|---|---|---|---|---|---|---|
| isoform | some | strand | number | start_coord | end_coord | category | site | canonical |
| PB.1087.18 | chr3 | — | junction_1 | 124905998 | 124906137 | novel | AGAG | non_canonical |
| PB.1087.18 | chr3 | — | junction_2 | 124906743 | 124908146 | known | GTAG | canonical |
| PB.1087.18 | chr3 | — | junction_3 | 124908349 | 124910414 | known | GTAG | canonical |
| PB.1087.18 | chr3 | — | junction_4 | 124910500 | 124912103 | known | GTAG | canonical |
| PB.1087.18 | chr3 | — | junction_5 | 124912142 | 124913110 | known | GTAG | canonical |
| PB.1087.18 | chr3 | — | junction_6 | 124913241 | 124913561 | known | GTAG | canonical |
| PB.1087.18 | chr3 | — | junction_7 | 124913682 | 124916316 | known | GTAG | canonical |
| PB.1087.18 | chr3 | — | junction_8 | 124916481 | 124920233 | known | GTAG | canonical |
| PB.1087.18 | chr3 | — | junction_9 | 124920290 | 124922196 | known | GTAG | canonical |
| PB.1087.18 | chr3 | — | junction_10 | 124922304 | 124923526 | known | GTAG | canonical |
| PB.1087.18 | chr3 | — | junction_11 | 124923650 | 124927531 | known | GTAG | canonical |
| PB.1087.18 | chr3 | — | junction_12 | 124927994 | 124934660 | known | GTAG | canonical |
| PB.1087.64 | chr3 | — | junction_1 | 124931778 | 124934586 | novel | CTAG | non_canonical |

INFLAMED

| | chromo- | | junction_ | genomic_ | genomic | junction_ | splice_ | |
|---|---|---|---|---|---|---|---|---|
| isoform | some | strand | number | start_coord | end_coord | category | site | canonical |
| PB.1087.6 | chr3 | — | junction_1 | 124906743 | 124908146 | known | GTAG | canonical |
| PB.1087.6 | chr3 | — | junction_2 | 124908349 | 124910414 | known | GTAG | canonical |
| PB.1087.6 | chr3 | — | junction_3 | 124910500 | 124912103 | known | GTAG | canonical |
| PB.1087.6 | chr3 | — | junction_4 | 124912142 | 124913161 | novel | GTAG | canonical |
| PB.1087.6 | chr3 | — | junction_5 | 124913241 | 124913561 | known | GTAG | canonical |
| PB.1087.6 | chr3 | — | junction_6 | 124913682 | 124916316 | known | GTAG | canonical |
| PB.1087.6 | chr3 | — | junction_7 | 124916481 | 124920233 | known | GTAG | canonical |
| PB.1087.6 | chr3 | — | junction_8 | 124920290 | 124922196 | known | GTAG | canonical |
| PB.1087.6 | chr3 | — | junction_9 | 124922304 | 124923526 | known | GTAG | canonical |
| PB.1087.63 | chr3 | — | junction_1 | 124931778 | 124934480 | novel | CAAG | non_canonical |
| PB.1087.21 | chr3 | — | junction_1 | 124906743 | 124908146 | known | GTAG | canonical |
| PB.1087.21 | chr3 | — | junction_2 | 124908349 | 124910414 | known | GTAG | canonical |
| PB.1087.21 | chr3 | — | junction_3 | 124910500 | 124912103 | known | GTAG | canonical |
| PB.1087.21 | chr3 | — | junction_4 | 124912142 | 124913110 | known | GTAG | canonical |
| PB.1087.21 | chr3 | — | junction_5 | 124913241 | 124913561 | known | GTAG | canonical |
| PB.1087.21 | chr3 | — | junction_6 | 124913682 | 124916316 | known | GTAG | canonical |
| PB.1087.21 | chr3 | — | junction_7 | 124916481 | 124920233 | known | GTAG | canonical |
| PB.1087.21 | chr3 | — | junction_8 | 124920290 | 124920708 | novel | GTAG | canonical |
| PB.1087.21 | chr3 | — | junction_9 | 124921025 | 124922196 | novel | GTAG | canonical |
| PB.1087.21 | chr3 | — | junction_10 | 124922304 | 124923526 | known | GTAG | canonical |
| PB.1087.21 | chr3 | — | junction_11 | 124923650 | 124927531 | known | GTAG | canonical |
| PB.1087.21 | chr3 | — | junction_12 | 124927994 | 124934660 | known | GTAG | canonical |
| PB.1087.20 | chr3 | — | junction_1 | 124906256 | 124913196 | novel | CAAG | non_canonical |
| PB.1087.20 | chr3 | — | junction_2 | 124913241 | 124913561 | known | GTAG | canonical |
| PB.1087.20 | chr3 | — | junction_3 | 124913682 | 124916316 | known | GTAG | canonical |
| PB.1087.20 | chr3 | — | junction_4 | 124916481 | 124920233 | known | GTAG | canonical |
| PB.1087.20 | chr3 | — | junction_5 | 124920290 | 124922196 | known | GTAG | canonical |
| PB.1087.20 | chr3 | — | junction_6 | 124922304 | 124923526 | known | GTAG | canonical |
| PB.1087.20 | chr3 | — | junction_7 | 124923650 | 124927531 | known | GTAG | canonical |
| PB.1087.20 | chr3 | — | junction_8 | 124927994 | 124934660 | known | GTAG | canonical |
| PB.1087.25 | chr3 | — | junction_1 | 124906743 | 124908146 | known | GTAG | canonical |
| PB.1087.25 | chr3 | — | junction_2 | 124908349 | 124910414 | known | GTAG | canonical |
| PB.1087.25 | chr3 | — | junction_3 | 124910500 | 124912103 | known | GTAG | canonical |
| PB.1087.25 | chr3 | — | junction_4 | 124912142 | 124913110 | known | GTAG | canonical |
| PB.1087.25 | chr3 | — | junction_5 | 124913203 | 124913577 | novel | AGAG | non_canonical |
| PB.1087.25 | chr3 | — | junction_6 | 124913682 | 124916316 | known | GTAG | canonical |
| PB.1087.25 | chr3 | — | junction_7 | 124916481 | 124920233 | known | GTAG | canonical |
| PB.1087.25 | chr3 | — | junction_8 | 124920290 | 124922196 | known | GTAG | canonical |
| PB.1087.25 | chr3 | — | junction_9 | 124922304 | 124923526 | known | GTAG | canonical |
| PB.1087.68 | chr3 | — | junction_1 | 124931778 | 124934705 | novel | CCAG | non_canonical |
| PB.1087.32 | chr3 | — | junction_1 | 124906743 | 124908146 | known | GTAG | canonical |
| PB.1087.32 | chr3 | — | junction_2 | 124908349 | 124913110 | novel | GTAG | canonical |
| PB.1087.32 | chr3 | — | junction_3 | 124913241 | 124913561 | known | GTAG | canonical |
| PB.1087.32 | chr3 | — | junction_4 | 124913682 | 124916316 | known | GTAG | canonical |
| PB.1087.32 | chr3 | — | junction_5 | 124916481 | 124920233 | known | GTAG | canonical |
| PB.1087.32 | chr3 | — | junction_6 | 124920290 | 124922196 | known | GTAG | canonical |
| PB.1087.32 | chr3 | — | junction_7 | 124922304 | 124923526 | known | GTAG | canonical |
| PB.1087.32 | chr3 | — | junction_8 | 124923650 | 124927531 | known | GTAG | canonical |
| PB.1087.32 | chr3 | — | junction_9 | 124927994 | 124934660 | known | GTAG | canonical |
| PB.1087.27 | chr3 | — | junction_1 | 124906225 | 124908173 | novel | AGAC | non_canonical |
| PB.1087.27 | chr3 | — | junction_2 | 124908349 | 124910414 | known | GTAG | canonical |
| PB.1087.27 | chr3 | — | junction_3 | 124910500 | 124912103 | known | GTAG | canonical |
| PB.1087.27 | chr3 | — | junction_4 | 124912142 | 124913110 | known | GTAG | canonical |
| PB.1087.27 | chr3 | — | junction_5 | 124913241 | 124913561 | known | GTAG | canonical |
| PB.1087.27 | chr3 | — | junction_6 | 124913682 | 124916316 | known | GTAG | canonical |
| PB.1087.27 | chr3 | — | junction_7 | 124916481 | 124920233 | known | GTAG | canonical |
| PB.1087.27 | chr3 | — | junction_8 | 124920290 | 124922196 | known | GTAG | canonical |
| PB.1087.27 | chr3 | — | junction_9 | 124922304 | 124923526 | known | GTAG | canonical |
| PB.1087.27 | chr3 | — | junction_10 | 124923650 | 124927531 | known | GTAG | canonical |
| PB.1087.27 | chr3 | — | junction_11 | 124927994 | 124934660 | known | GTAG | canonical |
| PB.1087.31 | chr3 | — | junction_1 | 124906743 | 124908146 | known | GTAG | canonical |
| PB.1087.31 | chr3 | — | junction_2 | 124908349 | 124910414 | known | GTAG | canonical |
| PB.1087.31 | chr3 | — | junction_3 | 124910500 | 124912103 | known | GTAG | canonical |
| PB.1087.31 | chr3 | — | junction_4 | 124912142 | 124913110 | known | GTAG | canonical |
| PB.1087.31 | chr3 | — | junction_5 | 124913241 | 124913561 | known | GTAG | canonical |
| PB.1087.31 | chr3 | — | junction_6 | 124913682 | 124916316 | known | GTAG | canonical |
| PB.1087.31 | chr3 | — | junction_7 | 124916481 | 124920233 | known | GTAG | canonical |
| PB.1087.31 | chr3 | — | junction_8 | 124920290 | 124922196 | known | GTAG | canonical |
| PB.1087.31 | chr3 | — | junction_9 | 124922304 | 124923526 | known | GTAG | canonical |
| PB.1087.31 | chr3 | — | junction_10 | 124923650 | 124927531 | known | GTAG | canonical |
| PB.1087.31 | chr3 | — | junction_11 | 124927874 | 124927951 | novel | AAAG | non_canonical |
| PB.1087.31 | chr3 | — | junction_12 | 124927994 | 124934660 | known | GTAG | canonical |
| PB.1087.52 | chr3 | — | junction_1 | 124922304 | 124923526 | known | GTAG | canonical |
| PB.1087.52 | chr3 | — | junction_2 | 124923650 | 124927531 | known | GTAG | canonical |
| PB.1087.52 | chr3 | — | junction_3 | 124927994 | 124934660 | known | GTAG | canonical |
| PB.1087.56 | chr3 | — | junction_1 | 124922624 | 124922693 | novel | GTGT | non_canonical |

### Example 3

### Aberrant Mucin Expression in Association With Tight Junction Dysfunction in the Respiratory and Intestinal Epithelium During SARS-CoV-2 Infection

### BACKGROUND

Severe acute respiratory syndrome coronavirus 2 (SARS-CoV-2), causing coronavirus disease 2019 (COVID-19), emerged in Wuhan, China, in December 2019. An initial cluster of infections was linked to the Huanan seafood market, potentially due to animal contact. SARS-CoV-2 is closely related to SARS-CoV, responsible for the SARS outbreak 18 years ago (Zhou et al., 2020), and has now spread rapidly worldwide. On March 11, 2020, the World Health Organization (WHO) declared COVID-19 a pandemic. Common symptoms reported in adults are fever, dry cough, fatigue and shortness of breath. While most COVID-19 patients (ca. 80%) remain asymptomatic or have mild to less severe respiratory complaints, some (ca. 15-20%) are hospitalised of which a minority develops a frequently lethal acute respiratory distress syndrome (ARDS). This results in mucus exudation, pulmonary oedema, hypoxia and lung failure in association with a cytokine storm characterized by amongst others Th17 immune profiles. Besides elderly or those with chronic underlying diseases, also young, healthy individuals die of COVID-19.

SARS-CoV-2 is a positive-sense single stranded RNA virus having 4 structural proteins, known as the S (spike), E (envelope), M (membrane) and N (nucleocapsid) proteins. The N protein holds the RNA genome, and the S, E and M proteins create the viral envelope. The S protein of coronaviruses regulates viral entry into target cells, i.e. ciliated epithelial cells. Entry depends on binding of the subunit Si to a cellular receptor, which facilitates viral attachment to the surface of target cells. Entry also requires S protein priming by cellular proteases, which cleave the S protein at its S1/S2 site allowing fusion of viral and cellular membranes, a process driven by the S2 subunit. Similar to SARS-CoV, the angiotensin-converting enzyme 2 (ACE2) is the entry receptor for SARS-CoV-2 and the cellular serine protease TMPRSS2 is essential for priming the S protein. ACE2 and TMPRSS2 expression is not only limited to the respiratory tract and extrapulmonary spread of SARS-COV-2 should therefore not be neglected. Indeed, a subset (ca. 30-35%) of COVID-19-positive patients (both ambulatory and hospitalised) showed gastrointestinal symptoms, including diarrhoea, abdominal pain, loss of appetite and nausea, and associated with a more indolent form of COVID-19 compared to patients with respiratory symptoms. Live SARS-CoV-2 was even successfully isolated from the stool of patients. This indicates that the intestinal epithelium is also susceptible to infection and recent work even provided evidence for an additional serine protease TMPRSS4 in priming the SARS-CoV-2S protein.

Furthermore, it has been suggested that the modest ACE2 expression in the upper respiratory tract has limited SARS-CoV transmissibility in the past. This is in large contrast to the currently reported SARS-CoV-2 infected cases which clearly surpassed that of SARS-CoV. In light of this increased transmissibility, we can speculate that this new coronavirus utilizes additional cellular attachment-promoting co-factors to ensure robust infection of ACE2⁺ cells in the respiratory tract. This could comprise binding to cellular glycans, as shown for other coronaviruses. Interestingly, mucus hyperproduction in the bronchioles and alveoli from severely ill COVID-19 patients has been reported (Guan et al., 2020; own observations ICU UZA), complicating the ICU stay and recovery. Secreted and transmembrane mucins are O-linked glycans produced by goblet and ciliated cells, respectively, and are the major components of the mucus layer covering the epithelial cells. Both mucus and epithelium constitute the mucosal barrier. Besides having a protective function, transmembrane mucins also participate in intracellular signal transduction and thus play an important role in mucosal homeostasis by establishing a delicate balance with tight junctions to maintain barrier integrity. Transmembrane mucins, particularly MUC13, might thus act as additional host factors enabling the virus to spread faster and cause tissue damage. In this study, we therefore investigated the expression patterns of ACE2, TMPRSS2/TMPRSS4, mucins and junctional proteins during SARS-CoV-2 infection in the respiratory and intestinal epithelium. Furthermore, the interplay between MUC13 and ACE2 expression upon viral infection was also studied.

### Material and Methods

### Viruses and Biosafety

The SARS-CoV-2 isolate 2019-nCoV/Italy-INMI1, available at the European Virus Archive-Global (EVAg) database, was used throughout the study. SARS-CoV-2 was subjected to passages in Vero E6 cells (green monkey kidney; ATCC CRL-1586), grown in Dulbecco's modified Eagle's minimal essential medium (DMEM; Gibco) supplemented with 10% heat-inactivated fetal calf serum (FCS), before usage in the cell culture experiments. The infectious viral titers in the cell-free supernatant were determined by a standard TCID5o assay. All experiments entailing live SARS-CoV-2 were conducted in the biosafety level 3 facility at the Institute for Tropical Medicine, Antwerp, Belgium.

### Cell Culture and Virus Infection

LS513 (human colorectal carcinoma (ATCC CRL-2134TM)) and Caco-2 (human colorectal carcinoma ATCC HTB-37) cells were grown in Roswell Park Memorial Institute (RPMI)-1640 medium (Life Technologies) supplemented with 10% heat-inactivated FCS, 100 U ml⁻¹ penicillin, 100 μg ml⁻¹ streptomycin, and 2 mM L-glutamine. Calu3 (lung adenocarcinoma ATCC HBT-55) cells were grown in Minimal Essential Medium (MEM; Gibco) supplemented with 10% heat-inactivated FCS, 100 U ml⁻¹ penicillin, 100 μg ml⁻¹ streptomycin, 1× MEM Non-essential Amino Acids and 1mM sodium pyruvate. For viral infection, all cells were seeded in 6 well-plates: 1×10⁶ cells/ml (LS513); 5×10⁵ cells/ml (Caco-2 and Calu3). After reaching confluence, the cells were inoculated with SARS-CoV-2 at a multiplicity of infection (MOI) of 0.1 for 24 h and 48 h at 37° C. (5% CO₂). Cells treated with the growth medium of the virus were included as controls. All experiments were performed containing 6 technical replicates for each time-point and cell line.

### Small Interfering RNA (siRNA) Transfection Assays

At the start of the transfection experiments, cells were seeded and grown in 6 well-plates (LS513: 1×10⁶ cells/ml; Caco-2 and Calu-3: 3×10⁵ cells/ml). After 24 hours, the cells were transfected with 75 pmol Silencer Select siRNA targeting MUC13 (s32232, ThermoFisher Scientific) or with 75 pmol Silencer Select Negative Control siRNA (4390843, ThermoFisher Scientific) using Lipofectamine RNAiMAX transfection reagent (7.5 μl/well, Invitrogen). Forty-eight hours post-transfection, cells were extensively washed and infected with SARS-CoV-2 at a MOI of 0.1 for 48 hours. Cells treated with the growth medium of the virus were included as controls. All transfection experiments were performed containing 6 technical replicates per cell line.

### RNA Extraction and Quantitative RT-PCR

Cells and supernatants were harvested at 24 hpi (hours post infection) and 48 hpi for quantitative RT-PCR analysis of host gene expression and virus replication, as previously described (Corman et al., 2020; Breugelmans et al., 2020). Briefly, total RNA from lysed cells and supernatants (100 μl) was extracted using the Nucleospin RNA plus kit (Macherey-Nagel) and QlAamp viral RNA kit (Qiagen), respectively, following the manufacturer's instructions. The concentration and quality of the RNA were evaluated using the Nanodrop ND-1000 UV-Vis Spectrophotometer (Thermo Fisher Scientific). For gene expression analysis, 1 μg RNA extracted from transfected and non-transfected cells was subsequently converted to cDNA by reverse transcription using the SensiFast™ cDNA synthesis kit (Bioline). Relative gene (i.e. ACE2, TMPRSS2, TMPRSS4, mucins and tight junctions) expression was then determined by SYBR Green RT-qPCR using the GoTaq qPCR master mix (Promega) on a QuantStudio 3 Real-Time PCR instrument (Thermo Fisher Scientific). Following quantitect primer assays (Qiagen) were used: Hs_GAPDH (QT00079247), Hs_ACTB (QT00095431), Hs_TMPRSS2 (QT00058156), Hs_TMPRSS4 (QT00033775), Hs_ACE2 (QT00034055), Hs_MUC1 (QT00015379), Hs_MUC2 (QT01004675), Hs_MUC4 (QT00045479), Hs_MUC5AC (QT00088991), Hs_MUC5B (QT01322818), Hs_MUC6 (QT00237839), Hs_MUC13 (QT00002478), Hs_CLDN1 (QT00225764), Hs_CLDN2 (QT00089481), Hs_CLDN3 (QT00201376), Hs_CLDN4 (QT00241073), Hs_CLDN7 (QT00236061), Hs_CLDN12 (QT01012186), Hs_CLDN15 (QT00202048), Hs_CLDN18 (QT00039550), Hs_CDH1 (QT00080143), Hs_OCLN (QT00081844), Hs_ZO-1 (QT00077308), Hs_ZO-2 (QT00010290).

All RT-qPCR reactions were performed in duplicate and involved an initial DNA polymerase activation step for 2 min at 95° C., followed by 40 cycles of denaturation at 95° C. for 15 sec and annealing/extension for 1 min at 60° C. Analysis and quality control were performed using qbase+ software (Biogazelle). Relative expression of the target genes was normalized to the expression of the housekeeping genes ACTB and GAPDH. To quantify viral RNA in the transfected and non-tranfected cells and supernatants, the iTaq Universal Probes One-Step kit (BioRad) was used on a LightCycler 480 Real-Time PCR System (Roche). A 25 μl reaction contained 1 μl RNA, 12.5 μl of 2×reaction buffer provided with the kit, 0.625 μl of iScript reverse transcriptase from the kit, 0.4 μl forward primer (25 μM), 0.4 μl reverse primer (25 μM), 0.5 μl probe (10 μM) targeting the SARS-CoV-2 E gene and 9.575 μl H₂O. We incubated the reactions at 50° C. for 10 min for reverse transcription, 95° C. for 5 min for denaturation, followed by 50 cycles of 95° C. for 10 s and 58° C. for 30 s. Analysis was performed using qbase+ software to determine cycle tresholds (Ct).

### Statistical Analysis

Statistical analysis using the GraphPad Prism 8.00 software (license DFG170003) was performed to determine significant differences between SARS-CoV-2 infected and uninfected cells and between MUC13 siRNA and ctrl siRNA transfected cells infected or not with SARS-CoV-2. Data were analysed by the Analysis of Variance (ANOVA) test and are presented as means±standard error of mean (SEM). Significance levels are indicated on the graphs and were corrected for multiple testing using the Tukey-Kramer's and Dunn's post-hoc multiple comparisons tests.

### Results and Discussion

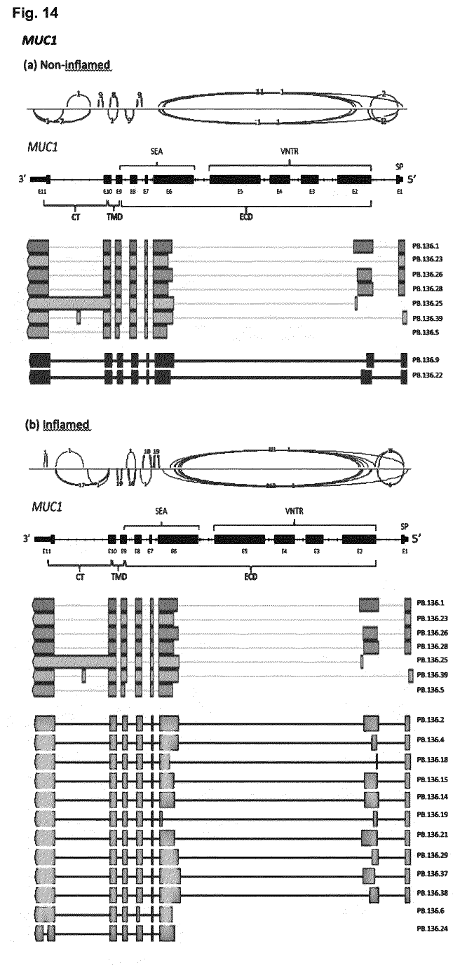

All cell lines tested here were susceptible for SARS-CoV-2 infection as shown by virus replication over a period of 48 h (data not shown). Virus production was significantly higher in the supernatant of Caco-2 and Calu3 cells compared to LS513 (p=0.0004; FIG. 16). This is in agreement with a recent study that described a robust replication of SARS-CoV-2 in both Caco-2 and Calu3 cells. Cell damage induced by SARS-CoV-2 was also assessed microscopically. No cytopathic effects, as typically described in Vero E6 cells, was noted in LS513 and Caco-2 cells. Interestingly, a substantial cell damage was noted in transfected Calu3 cells (30% viability at 48 hpi; p<0.001) but not in non-transfected cells. The induction of cell damage in Calu3 cells caused by corona viruses still remains controversial. A recent study described no cell death in SARS-CoV- and SARS-CoV-2-infected Calu3 cells, whereas earlier studies reported that SARS-CoV infection induced cytopathic effects in Calu3. A reason for these discrepancies is currently unknown, but it cannot be excluded that in our study transfection with siRNA made the cells more susceptible for viral cytopathic effects.

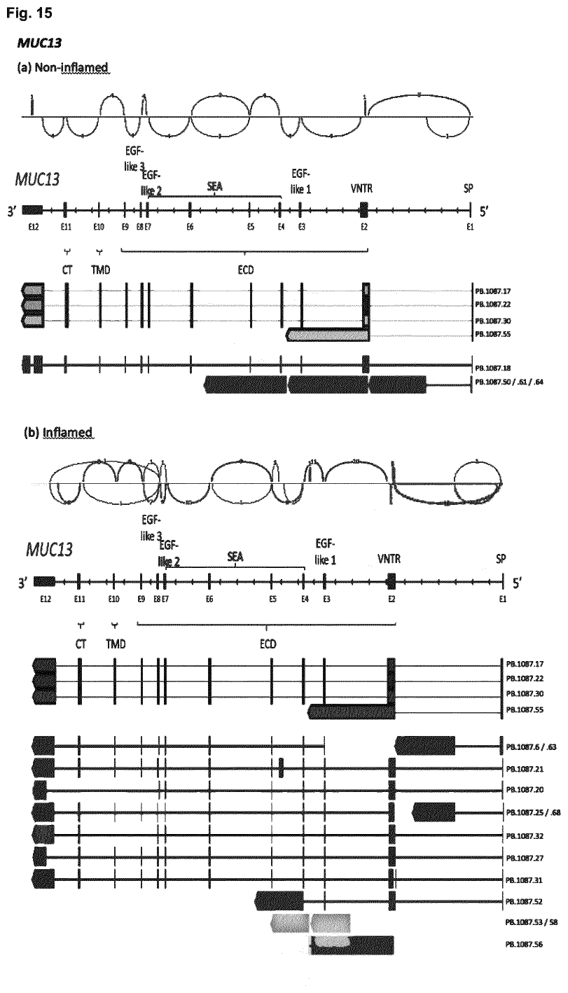

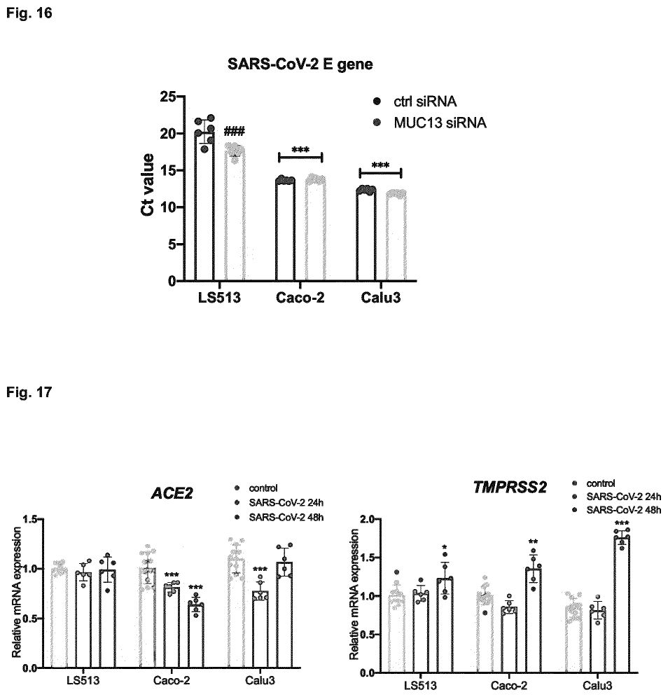

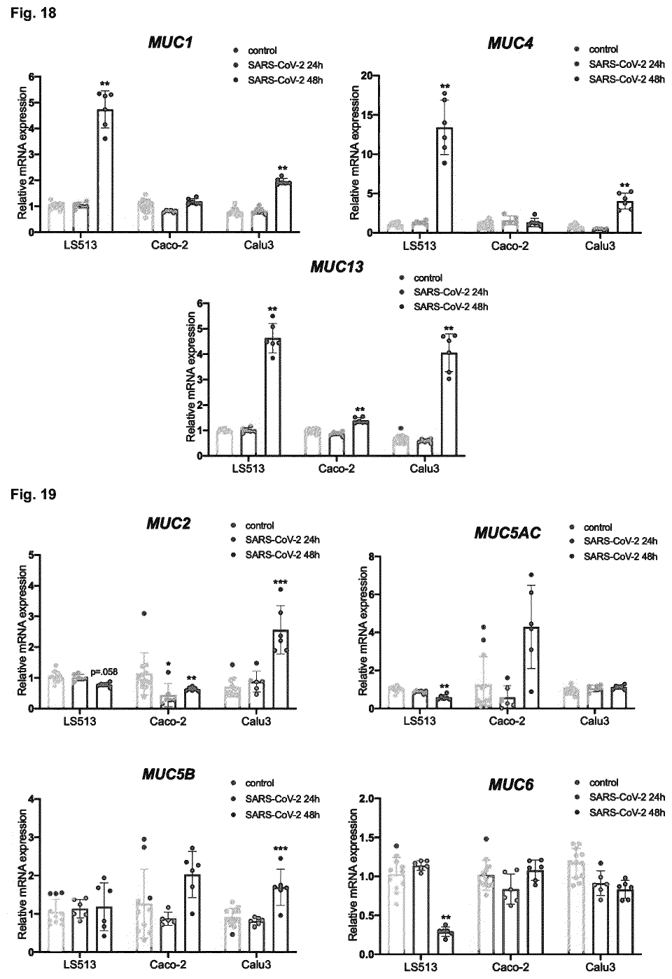

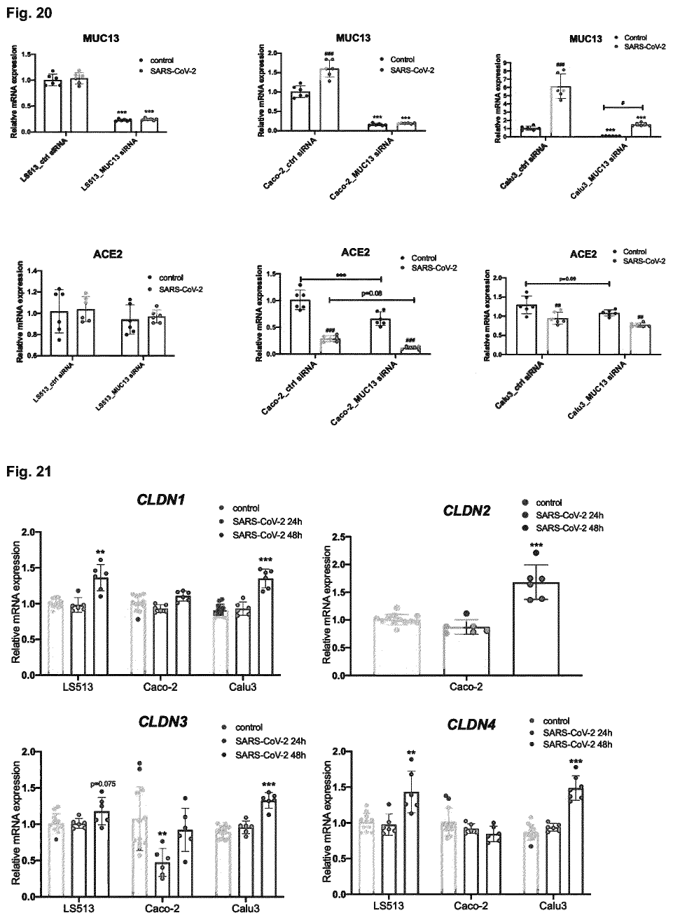

As SARS-CoV-2 uses the receptor ACE2 for entry and the serine proteases TMPRSS2 and TMPRS S4 for S protein priming, expression of these host factors was investigated. In our study, ACE2 mRNA expression was significantly reduced in Caco-2 cells at 24 hpi (p=0.0001) and 48 hpi (p=0.0008) and in Calu3 cells at 24 hpi only (p=0.0004) (FIG. 17). No changes in ACE2 expression were noted in LS513 which could explain the significant lower virus production compared to Caco-2 and Calu3 (FIGS. 16 & 17). ACE2 is an important component of the renin-angiotensin pathway and counterbalances the effects of AT1 activation by angiotensin II. In the lungs, ACE2 has an anti-inflammatory role protecting the respiratory tract from injury, whereas it maintains mucosal barrier homeostasis in the intestines by regulating expression of antimicrobial peptides (AMPs) and the ecology of the gut microbiome. Downregulation of this receptor upon SARS-CoV-2 infection could thus exaggerate acute lung and intestinal injury because of the imbalance in angiotensin II or AT1 signalling. On the contrary, expression of TMPRSS2 was significantly increased in all cell types at 48 hpi (TMPRSS2: p=0.0433 (LS513), p=0.0057 (Caco-2), p=0.0001 (Calu3); FIG. 17) compared to uninfected controls whereas upregulation of TMPRSS4 was remarkably only seen in Calu3 cells (p=0.0152). The abundancy of TMPRSS2 and to a lesser extend TMPRSS4 is thus essential for promoting viral entry into host cells. In addition, TMPRSS2 is also an important mediator of mucosal barrier dysfunction and linked to aberrant mucin expression. We therefore also investigated the impact of SARS-CoV-2 infection on mucin and tight junction expression. In our study, significant changes in mucin expression were mainly seen at 48 hpi. More specifically, the transmembrane MUC1, MUC13 and MUC4 mucins were strongly upregulated in both intestinal and pulmonary SARS-CoV-2-infected epithelial cells (MUC1: p=0.0022 (LS513); p=(Calu3); MUC4: p=0.0022 (LS513); p=0.0022 (Calu3); MUC13: p=0.0022 (LS513); p=0.0022 (Caco-2); p=0.0022 (Calu3); FIG. 18), whereas the secreted mucins (particularly MUC2 (p=0.058 (LS513); p=(Caco, 24 hpi); p=(Caco-2; 48 hpi)), MUC5AC (p=0.0012 (LS513)) and MUC6 (p=0.0022 (LS513)), which are at the frontline of mucosal defence (Linden et al., 2007), were significantly downregulated with the exception of MUC2 (p=0.0001) and MUCSAB (p=0.0001) expression in Calu3 cells (FIG. 19). As own data showed a functional link between MUC13 and ACE2, we further investigated whether ACE2 downregulation upon viral infection is mediated by MUC13 using siRNA transfection assays. Knockdown of MUC13 was successful in all three cell lines in which a reduction in MUC13 expression of approximately 70-80% was maintained during infection (FIG. 5). In ctrl siRNA transfected Caco-2 and Calu-3 cells, MUC13 expression significantly increased upon SARS-CoV-2 infection whereas ACE2 expression significantly decreased (FIG. 20). This is in agreement to what is seen in wildtype SARS-CoV-2-infected Caco-2 and Calu3 cells (FIG. 18). Interestingly, knockdown of MUC13 decreased ACE2 expression in Caco-2 and Calu3 control cells (p=0.0004 (Caco-2); p=0,09 (Calu3)) and its expression further declined upon SARS-CoV-2 infection although not significant (FIG. 20). This strengthens the evidence that ACE2 expression is mediated by MUC13. In addition, MUC13 expression was not altered in ctrl siRNA transfected LS513 cells upon infection (FIG. 20) which is in contrast to what is seen in wildtype SARS-CoV-2-infected LS513 cells (FIG. 18). ACE2 expression remained unchanged (FIG. 20) and lower virus production in the supernatants was noted (FIG. 16). This further highlights the importance of increased MUC13 expression mediating ACE2 signalling for optimal virus production.

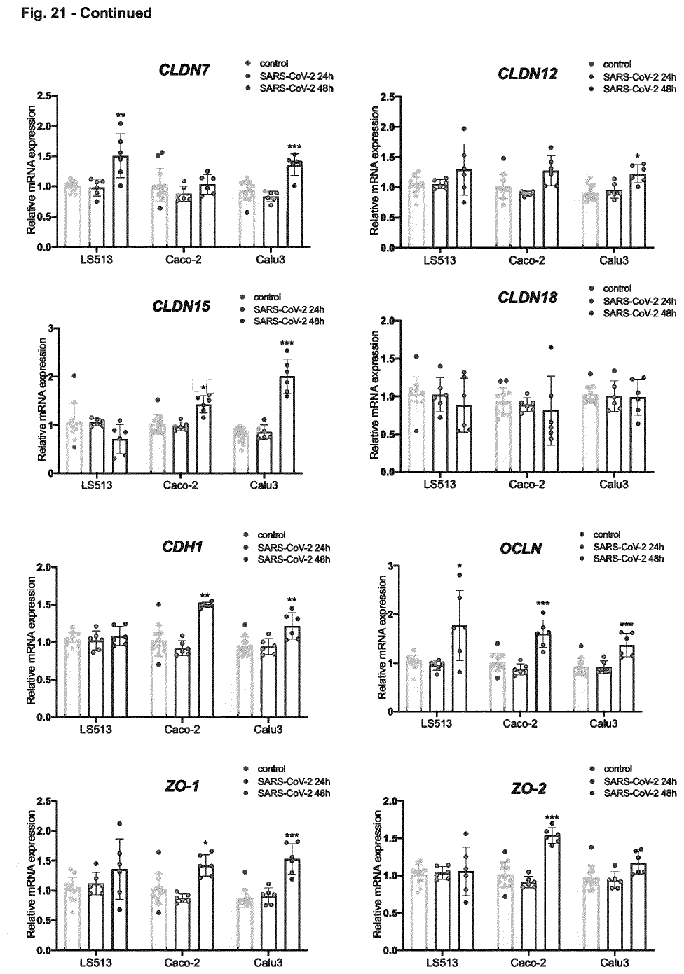

Furthermore, inappropriate overexpression of MUC13 can also affect barrier integrity by disrupting cell polarity and cell-cell interactions resulting in tight junction dysfunction, as recently shown. In our study, a significant increase in gene expression of several junctional proteins was noted at 48 hpi (FIG. 21), suggesting the ability of SARS-CoV-2 to alter epithelial barrier integrity, as described for SARS-CoV. Most alterations in expression were seen in LS513 and Calu3 cells, i.e.: CLDN1 (p=0.0022 5LS513); p=0.0001 (Calu3)), CLDN2 (p=0.0007 (Caco-2)), CLDN3 (p=0.075 (LS513); p=0.0001 (Calu3)), CLDN4 (p=0.01 (LS513); p=0.0001 (Calu3)), CLDN7 (p=0.0085 (LS513); p=0.0001 (Calu3)), CLDN12 (p=0.031 (Calu3)), CLDN15 (p=0.0139 (Caco-2); P=0.0004 (Calu3)), CDH1 (p=0.003 (Caco-2); p=0.0013 (Calu3)), OCLN (p=0.0335 (LS513); p=0.0004 (Caco-2); p=0.0002 (Calu3)), ZO-1 (p=0.034 (Caco-2); p=0.0001 (Calu3)) and ZO-2 (p=0.0005 (Caco-2)).

Taken together, the results from this study further underline the tropism of SARS-CoV-2 for both the respiratory and intestinal epithelium and demonstrate that this novel coronavirus strongly affects the mucosal barrier integrity upon infection by inducing aberrant mucin expression and tight junction dysfunction. Furthermore, a role for transmembrane mucins, particularly MUC13, in contributing to the infection of SARS-CoV-2 is also suggested.

### REFERENCES

Breugelmans T, Van Spaendonk H, De Man J G, De Schepper H U, Jauregui-Amezaga A, Macken E, Linden S K, Pintelon I, Timmermans J P, De Winter B Y, Smet A. In depth study of transmembrane mucins in association with intestinal barrier dysfunction during the course of T cell transfer and DSS-induced colitis. J Crohns Colitis 2020, jjaa015.

Corman V M, Landt O, Kaiser M, Molenkamp R, Meijer A, Chu D K, Bleicker T, Brünink S, Schneider J, Schmidt M L, Mulders D G, Haagmans B L, van der Veer B, van den Brink S, Wijsman L, Goderski G, Romette J L, Ellis J, Zambon M, Peiris M, Goossens H, Reusken C, Koopmans M P, Drosten C. Detection of 2019 novel coronavirus (2019-nCoV) by real-time RT-PCR. Euro Surveill 2020, 25(3):2000045.

Guan W J, Chen R C, Zhong N S. Strategies for the prevention and management of coronavirus disease 2019. Eur Respir J. 2020, 55(4):2000597.

Gupta J, Nebreda A. Analysis of Intestinal Permeability in Mice. BIO-PROTOCOL 2014; 4(22).

Heylen M, Deleye S, De Man J G, et al. Colonoscopy and μPET/CT are Valid Techniques to Monitor Inflammation in the Adoptive Transfer Colitis Model in Mice. Inflamm Bowel Dis 2013; 19(5): 967-76.

Linden S, Putton P, Karlsson N, et al. Mucins in the mucosal barrier to infection. Mucosal Immunol 2008; 1(3): 183-97.

Moehle C, Ackermann N, Langmann T, et al. Aberrant intestinal expression and allelic variants of mucin genes associated with inflammatory bowel disease. J Mol Med 2006; 84(12): 1055-66.

Moreels T G, Nieuwendijk R J, De Man J G, et al. Concurrent infection with Schistosoma mansoni attenuates inflammation induced changes in colonic morphology, cytokine levels, and smooth muscle contractility of trinitrobenzene sulphonic acid induced colitis in rats. Gut 2004; 53(1): 99-107.

Obermair A, Schmid B C, Packer L M, Leodolter S, Birner P, Ward B G, Crandon A J, McGuckin M A, Zeillinger R. Expression of MUC1 splice variants in benign and malignant ovarian tumours. Int J Cancer 2002; 100: 166-71.

Sheng Y H, Lourie R, Lindén S K, et al. The MUC13 cell-surface mucin protects against intestinal inflammation by inhibiting epithelial cell apoptosis. Gut 2011; 60(12): 1661-70.

Sheng Y, Triyana S, Wang R, et al. MUC1 and MUC13 differentially regulate epithelial inflammation in response to inflammatory and infectious stimuli. Mucosal Immunol 2012; 6(3): 557-68.

Vancamelbeke M, Vanuytsel T, Farré R, et al. Genetic and Transcriptomic Bases of Intestinal Epithelial Barrier Dysfunction in Inflammatory Bowel Disease. Inflamm Bowel Dis 2017; 23(10): 1718-29.

Wallace J L, Keenan C M, Gale D, Shoupe T S. Exacerbation of experimental colitis by nonsteroidal anti-inflammatory drugs is not related to elevated leukotriene B4synthesis. Gastroenterology 1992; 102(1): 18-27.

Zaretsky J Z, Barnea I, Aylon Y, Gorivodsky M, Wreschner D H, Keydar I. MUC1 gene overexpressed in breast cancer: Structure and transcriptional activity of the MUC1 promoter and role of estrogen receptor alpha (ERα) in regulation of the MUC1 gene expression. Mol Cancer 2006; 5: 57.

Zrihan-Licht S, Vos H L, Baruch A, Elroy-Stein O, Sagiv D, Keydar I, Hilkens J, Wreschner D H. Characterization and Molecular Cloning of a Novel MUC1 Protein, Devoid of Tandem Repeats, Expressed in Human Breast Cancer Tissue. Eur J Biochem 1994; 224: 787-95.

1-11. (canceled)

12. A method for diagnosing, monitoring, and/or treating a disease characterized by barrier dysfunction, the method comprising:
providing a biological sample from a subject;
determining an expression level of at least one mucin isoform in the biological sample, wherein the mucin isoform is selected from MUC1 isoforms or MUC13 isoforms; and
comparing the expression level to a control, wherein an increased expression level compared to the control is indicative of the disease.

13. The method according to claim 12, wherein determining an expression level comprises determining an mRNA expression level.

14. The method according to claim 12, wherein determining an expression level comprises immunohistochemically staining the biological sample and measuring the stain intensity.

15. The method according to claim 14, wherein immunohistochemically staining comprises contacting the sample with at least one antibody selective for at least one mucin isoform.

16. The method according to claim 12, wherein the mucin isoform is a transmembrane mucin.

17. The method according to claim 12, further comprising administering a treatment targeting the mucin isoform.

18. The method according to claim 17, wherein the treatment comprises monoclonal antibodies, small molecules, or antisense therapy.

19. The method according to claim 12, wherein the disease characterized by barrier dysfunction comprises a gastrointestinal disorder, a neurodegenerative disorder, or a respiratory infection.

20. The method according to claim 19, wherein the disease characterized by barrier dysfunction is a gastrointestinal disorder selected from the group consisting of inflammatory bowel disease, irritable bowel syndrome, cancer, gastrointestinal infections, obesitas, and non-alcoholic fatty liver disease.

21. The method according to claim 20, wherein the gastrointestinal disorder is a cancer selected from the group consisting of esophageal cancer, gastric cancer, colorectal cancer, pancreas cancer, liver cancer, kidney cancer, lung cancer, ovarian cancer, colon cancer, and prostate cancer.

22. The method according to claim 20, wherein the gastrointestinal disorder is a gastrointestinal infection selected from the group consisting of Helicobacter infection, Campylobacter infection, Clostridioides difficile infection and Salmonella infection.

23. The method according to claim 20, wherein the gastrointestinal disorder is an inflammatory bowel disease selected from the group consisting of Crohn's disease and ulcerative colitis.

24. The method according to claim 19, wherein the disease characterized by barrier dysfunction is a neurodegenerative disorder selected from the group consisting of Parkinson's disease, Alzheimer's disease, multiple sclerosis, and autism.

25. The method according to claim 19, wherein the disease characterized by barrier dysfunction is a respiratory infection selected from the group consisting of respiratory syncytial viral infections, influenza viral infections, rhinoviral infections, metapneumoviral infections, Pseudomonas aeruginosa viral infections, and coronaviral infections.

26. The method according to claim 25, wherein the respiratory infection is a coronaviral infection.

27. The method according to claim 26, wherein the coronaviral infection is a SARS-CoV-2 infection.
